(19) United States
(12) Patent Application Publication
(10) Pub. No.: US 2023/0158116 A1
(43) Pub. Date: May 25, 2023

(54) USE OF HUMAN AMYLIN ANALOG POLYPEPTIDES FOR PROVIDING SUPERIOR GLYCEMIC CONTROL TO TYPE 1 DIABETICS

(71) Applicant: I2O Therapeutics, Inc., Cambridge, MA (US)

(72) Inventor: Andrew YOUNG, Cambridge, MA (US)

(21) Appl. No.: 17/996,131

(22) PCT Filed: Apr. 20, 2021
(86) PCT No.: PCT/US2021/028209

§ 371 Date: Oct. 13, 2022

Related U.S. Application Data
(60) Provisional application No. 63/012,619, filed on Apr. 20, 2020.

(51) Int. Cl. A61K 38/22; A61K 38/28; A61K 9/00; A61P 3/10

(52) U.S. Cl. CPC A61K 38/22; A61K 38/28; A61K 9/0019; A61P 3/10

(57) ABSTRACT

This invention relates to the administration of analogs of human amylin for the treatment of type 1 diabetes. Methods described herein augment insulin injection or infusion therapy with separate and continuous co-administration of an amylin analog at a therapeutically effective dose of at least 5 mg per kilogram per day or at a therapeutically effective dose that is equivalent to at least ED70 dose of the amylin analog, as defined herein.

### CROSS-REFERENCE TO RELATED APPLICATION

This application claims the benefit of U.S. Provisional Patent Application No. 63/012,619, filed Apr. 20, 2020, the disclosure of which is incorporated herein by reference in its entirety.

### REFERENCE TO SEQUENCE LISTING SUBMITTED ELECTRONICALLY

This application contains a sequence listing, which is submitted electronically via EFS-Web as an ASCII formatted sequence listing with a file name “Seq-Listing-717156_102487-060WO.txt,” creation date of Apr. 17, 2021 and having a size of 4 KB. The sequence listing submitted via EFS-Web is part of the specification and is herein incorporated by reference in its entirety.

### FIELD

This disclosure relates to treatments of type 1 diabetes.

### BACKGROUND

Type 1 diabetes is a devastating disease. Type 1 diabetics lack functional pancreatic β-cells and, as such, cannot produce insulin and amylin that are otherwise secreted from functional beta cells of relatively healthy individuals. Type 1 diabetics require self-injections of insulin to survive. However, such self-injections of insulin can be difficult to manage to avoid and minimize potentially adverse, even life-threatening, side effects associated with hypoglycemia. There is a significant need for improved therapies for type 1 diabetes.

Loss of β-cell function that occurs early in type 1 diabetics and can occur late in type 2 diabetics leads to deficiencies in the secretion of insulin and amylin.

Insulin is a peptide that regulates blood glucose levels and coordinates the body's distribution and uptake of glucose. Insulin's role in the body is, among other things, to prevent blood glucose levels from rising too high, particularly after a meal.

Human amylin, or islet amyloid polypeptide (IAPP), is a 37-residue polypeptide hormone. Pro-islet amyloid polypeptide (i.e., pro-IAPP) is produced in the pancreatic β-cells as a 67 amino acid, 7404 Dalton pro-peptide that undergoes post-translational modifications including protease cleavage to produce the 37-residue amylin. Amylin is co-secreted with insulin from pancreatic β-cells in the ratio of approximately 100:1 (insulin:amylin). Amylin and insulin levels rise and fall in a synchronous manner. Amylin and insulin have complementary actions in regulating nutrient levels in the circulation. Whereas insulin aids and promotes storage of nutrients, amylin slows nutrient entry/storage in the body.

Amylin functions as part of the endocrine pancreas, those cells within the pancreas that synthesize and secrete hormones. Amylin contributes to glycemic control; it is secreted from the pancreatic islets into the blood circulation and is cleared by peptidases in the kidney. Amylin's metabolic function is well-characterized as an inhibitor of the appearance of nutrients, such as glucose, in the plasma. It thus functions as a synergistic partner to insulin, which regulates blood glucose levels and coordinates the body's distribution and uptake of glucose.

Amylin is believed to play a role in glycemic regulation by slowing gastric emptying and promoting satiety (i.e., feeling of fullness), thereby preventing post-prandial (i.e., after-meal) spikes in blood glucose levels. The overall effect is to slow the rate of appearance of glucose in the blood after eating. Amylin also lowers the secretion of glucagon by the pancreas. Glucagon's role in the body is, among other things, to prevent blood glucose levels dropping too low. This is significant because certain type 1 diabetics, for example, are prone to secrete excess amounts of the blood glucose-raising glucagon just after meals.

For numerous reasons, human amylin, having a half-life in serum of about 13 minutes, is not amenable for use as a therapeutic agent. Rather, pramlintide was developed as a synthetic analogue of human amylin (i.e., amylin receptor agonist) for the treatment of patients with types 1 or 2 diabetes, who use meal-time insulin but cannot achieve desired glycemic control despite optimal insulin therapy. Pramlintide differs from human amylin in 3 of its 37 amino acids. These modifications reduce its propensity to aggregate, a characteristic found of human amylin.

For treatment of type 1 diabetics, pramlintide is administered up to four times per day, via subcutaneous injection before meals, as an adjunct to insulin therapy administered after meals. Pramlintide cannot be mixed with insulin; separate syringes are used. Reported side effects of pramlintide include nausea and vomiting. Adverse reactions can include severe hypoglycemia, particularly for type 1 diabetics. Consequently, dosage of meal-time insulin is reduced for patients who initiate administration of pramlintide.

Accordingly, there exists a need for improved methods of administration of amylin analog polypeptides in conjunction with insulin to provide enhanced glycemic control among type 1 diabetics, and particularly for avoiding an onset of insulin-induced hypoglycemia (including iatrogenic hypoglycemia).

### SUMMARY

Applicants have discovered a series of potential solutions for improved treatment of type 1 diabetes as described herein:

Specifically, Applicants have discovered treatment regimens that shift a so-called “burden for treatment” (as defined below) for glucose control in type 1 diabetics from insulin to an amylin agonist. In essence, methods herein describe methods for (i) continuous administration and (ii) a high therapeutically effective dose of the amylin analog. By providing the type 1 diabetic with continuous administration and a high therapeutically effective dose of the amylin analog, less insulin is required to control blood sugar concentrations and increase time-in-range for the patient (i.e., the length of time during which a type 1 diabetic patient maintains serum glucose concentrations of approximately 70 mg/dL to 180 mg/dL.) As such, lower doses of insulin can separately be provided because control of blood glucose is largely provided by the amylin agonist.

Continuous administration of the amylin analog: For example, to achieve continuous administration, the amylin analog is administered to the patient via an implantable (e.g., osmotic) or non-implantable (external infusion pump) drug delivery device. Both short-acting amylin analogs (e.g., pramlintide) or long acting amylin analogs (e.g., compound A2, described herein) can be administered to the patient via an implantable (e.g., osmotic) or non-implantable (external infusion pump) drug delivery device to achieve continuous administration. Further, continuous presence of a long-acting amylin analog (e.g., compound A2) can also be achieved in the patient by administration via infrequent (e.g., once weekly) injections.

As used herein, “short-acting amylin analog” has an elimination half-life (t_{1/2}) of twelve hours or less and a “long-acting amylin analog” has an elimination half-life (t_{1/2}) of greater than twelve hours.

High therapeutically effective dose of the amylin analog: Methods are provided for administration to a patient of an amylin analog at a “high” therapeutically effective dose, for example, of at least 5 μg per kilogram per day. In certain embodiments, methods are provided for administering to a patient an amylin analog at a high therapeutically effective dose of at least 10 μg per kilogram per day, 50 μg per kilogram per day, or 100 μg per kilogram per day.

Alternatively, a high therapeutically effective dose of the amylin analog is achieved upon administration of a dose corresponding to at least the ED70 dose of the amylin agonist. In some embodiments, methods herein employ a therapeutically effective dose that is at least the ED75, ED80, ED85, ED90 or ED95 dose of the amylin agonist.

As used herein, the term “ED70 dose” (or ED75, ED80, ED85, ED90 or ED95 dose) refers to a dose regimen that results in a plasma drug concentration sufficient to activate amylin receptors, also referred to herein as amylin response(s), to a level that is 70% (or 75%, 80%, 85%, 90% or 95%, respectively) of the maximum attainable response. In some embodiments, the amylin analog is an agent that activates a heterodimeric receptor constituted from a calcitonin receptor and a RAMP3 (receptor activity modulating peptide 3), also known as an amylin 3 receptor. In some embodiments, the amylin 3 receptor is a human amylin 3 receptor.

Known methods for the treatment of type 1 diabetes with insulin and an amylin agonist (pramlintide) have been deficient in this regard because such methods neither provide (i) continuous administration of the amylin analog (e.g., via an implantable or non-implantable drug delivery device), nor do they provide (ii) a high therapeutically effective dose of the amylin analog (e.g., of at least 5 μg per kilogram per day; or a therapeutically effective dose that is at least the ED70 dose of the amylin agonist).

Specifically, methods have been discovered for administration of analogs of human amylin that provides relatively continuous steady-state exposure of the amylin analog to provide enhanced glycemic control among type 1 diabetics relative to either (i) insulin therapy alone or (ii) insulin therapy in conjunction with daily (or up to 4 times daily) injectable administration of a short-acting analog of human amylin, such as pramlintide (Symlin®, developed by Amylin Pharmaceuticals, Inc., San Diego, Calif., USA and marketed by AstraZeneca plc, Cambridge, UK). According to the methods disclosed herein, relatively continuous steady-state exposure of analogs of human amylin is achieved by (i) administration via implantable drug delivery device of a long-acting amylin analog (such as compound A2 described herein) or a short-acting amylin analog (such as pramlintide) or (ii) administration via infrequent (e.g., once weekly) injection of a long-acting amylin analog such as compound A2.

As such, the disclosure provides methods for maintaining glycemic control (e.g., maintaining normoglycemia) in type 1 diabetics in need thereof, and particularly for avoiding (or minimizing the likelihood of) an onset of insulin-induced hypoglycemia (including iatrogenic hypoglycemia).

One aspect of the disclosure provides a method of maintaining glycemic control (e.g., maintaining normoglycemia or treating iatrogenic hypoglycemia) among type 1 diabetics, comprising; continuously administering a high therapeutically effective dose of an amylin agonist, such as compound A2 or pramlintide, via (i) infusion, (ii) once weekly injection, (iii) an implantable drug delivery device or (iv) a non-implantable drug delivery device. In some embodiments, the method further comprises separate administration of an insulin, such as a long-acting insulin.

There are no known reports that an amylin analog has been delivered clinically as a continuous infusion. Instead, clinical delivery has been a basal dose in association with a meal-related bolus doses, with the latter constituting the majority of drug delivered. Furthermore, current treatment regimens are focused on physiological concentrations of amylin and their ratio to insulin based on limitations inherent to amylin bolus dosing.

Despite the known therapeutic value of amylin analogs, and amylin analog (e.g., pramlintide)-insulin combinations, current treatment modes are limited in a number of aspects. Chiefly, current amylin-insulin therapy does not adequately shift the burden of glucose control onto glucose-sensitive amylin-analog mediated signaling. Furthermore, current treatment modes are limited to unpleasant bolus administration and attendant side-effects (e.g. nausea), and the need for careful blood-glucose monitoring. Accordingly, there is a need in the art for improved methods of administration of amylin analogs, particularly in conjunction with insulin to provide greater glycemic control and therapeutic outcomes.

### BRIEF DESCRIPTION OF THE DRAWINGS

FIG. 1 illustrates, as described in Example 1, cumulative distribution of blood glucose values before (black line) and alter (red line) treatment with STZ. Post-STZ values related to animals treated with Levemir insulin, ˜1, 2 or 6 U/day, n=5. The range 70-180 mg/dL is shown by the vertical dotted lines, 98.5% of pre-STZ values fell within the range (TIR=98.8%). The value post-STZ treated with 2 U/day Levemir was 64.9%; 5.8% of values were <70 mg/dL.

FIG. 2 illustrates that amylin (or an amylin analog) and insulin share the burden of plasma glucose control. Amylin (or an amylin analog) provides a unique opportunity for plasma glucose control that is active only during periods of elevated plasma glucose levels. At a sufficiently high dose of an amylin analog, glucose-dependent effects of amylin agonism will be able to substitute for the glucose-independent effects of insulin. Accordingly, the resulting diminished requirement for insulin will reduce the risk of “overshooting” an insulin dosage and its unintended consequence to patients of treatment-induced hypoglycemia.

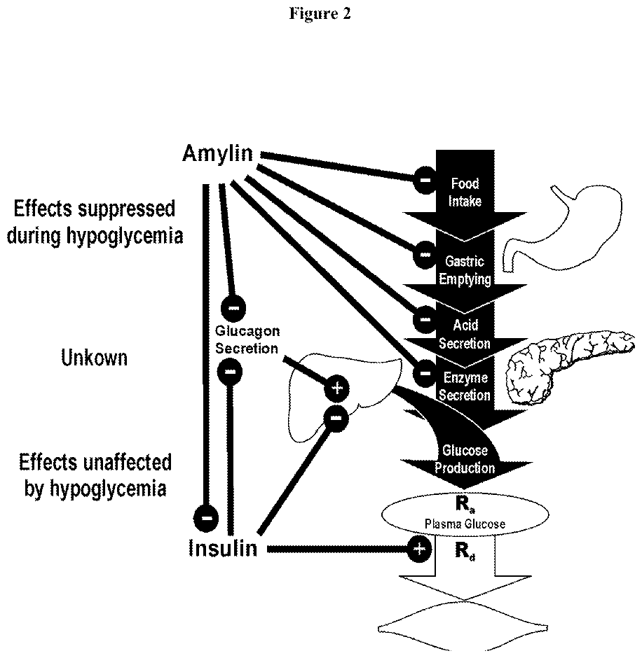

FIG. 3 illustrates a therapeutic goal of the disclosed methods of treatment such as a reduction in hypoglycemic events. Microvascular benefits of lower mean glucose have not been achievable via FDA-approved therapies at present because, in part, of incipient iatrogenic hypoglycemia from bolus insulin. Lower propensity to hypoglycemia would allow lower glycemic equipoise.

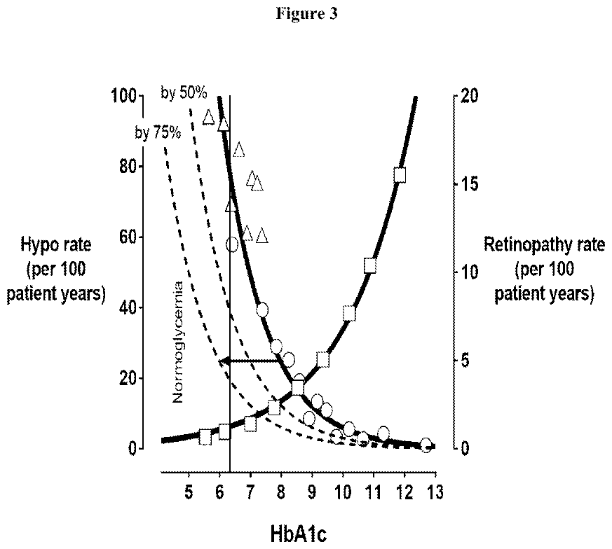

FIG. 4 illustrates a therapeutic goal of the disclosed methods of treatment such as a reduction in glucose excursions. Amylin agonism offers modes of glucose regulation that are distinct from those achieved with insulin. Importantly, amylin action is glucose-dependent

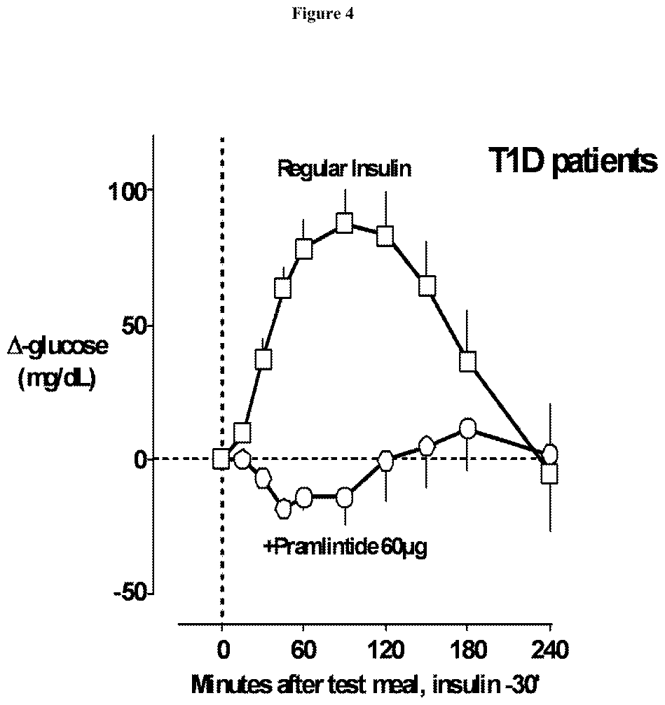

FIG. 5 illustrates certain advantages of continuous delivery of a long acting amylin analog (i.e., agonist) administered via an implanted device (e.g., osmotic mini pump) in conjunction with supplemental insulin therapy. Under the Typical Profile, multiple daily injections of insulin & amylin analog are required. Dosing is titrated meal-by-meal in response to glucose measures. Relatively high insulin to amylin analog ratio places the therapeutic burden on glucose independent insulin. Under the Developed Profile, fixed doses of the following are delivered: (i) a short or long acting amylin analog via an implanted device (e.g., osmotic mini pump) or (ii) a long acting amylin analog via infrequent (e.g., weekly) injections. Relatively lower doses of insulin may diminish the risk for side effects such as hypoglycemia. Relatively high amylin analog to insulin ratio places the therapeutic burden on glucose dependent hormone amylin.

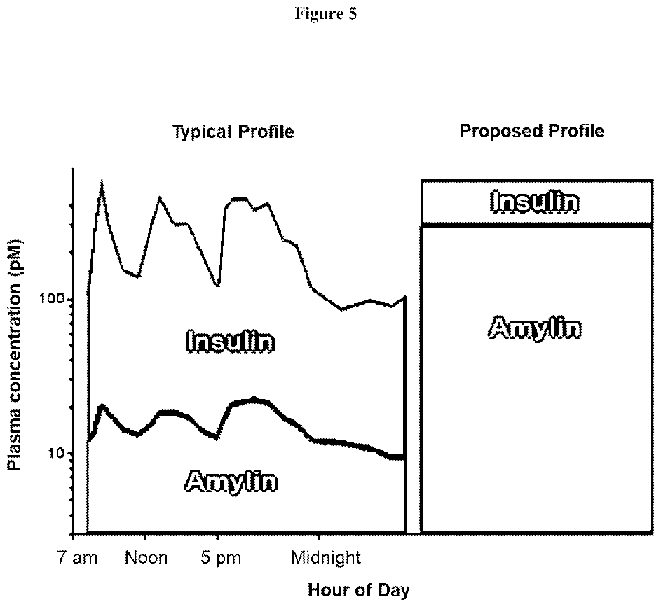

FIG. 6 depicts a clinical study plan to compare pramlintide injection vs infusion (constant delivery). “CV” means Clinic Visit for data download and subject training. “PK” means collection of a pramlintide PK sample. Contact with patient to be scheduled as needed during each titration period.

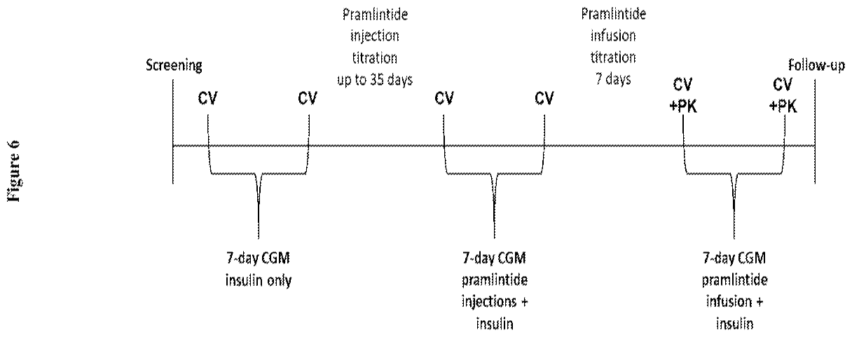

### DETAILED DESCRIPTION

### General Description of Certain Embodiments of the Disclosure

This disclosure relates to methods of using amylin analogs for treating metabolic diseases or disorders, such as types 1 and 2 diabetes, obesity, and methods of providing weight loss.

### Definitions

It is to be understood that the terminology used herein is for the purpose of describing particular embodiments only and is not intended to be limiting. As used in this specification and the appended claims, the singular forms “a,” “an” and “the” include plural referents unless the context clearly dictates otherwise. Thus, for example, reference to “a solvent” includes a combination of two or more such solvents, reference to “a peptide” includes one or more peptides, or mixtures of peptides, reference to “a drug” includes one or more drugs, reference to “an osmotic delivery device” includes one or more osmotic delivery devices, and the like. Unless specifically stated or obvious from context, as used herein, the term “*or” is understood to be inclusive and covers both “or” and “and”.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the term “substantially” is understood as within a narrow range of variation or otherwise normal tolerance in the art. Substantially can be understood as within 5%, 4%, 3%, 2%, 1%, 0.5%, 0.1%, 0.05%, 0.01% or 0.001% of the stated value.

Unless defined otherwise, all technical and scientific terms used herein have the same meaning as commonly understood by one of ordinary skill in the art to which the disclosure pertains. Although other methods and materials similar, or equivalent, to those described herein can be used in the practice of the present disclosure, the preferred materials and methods are described herein.

The following terminology will be used in accordance with the definitions set out below.

The terms “burden” and “therapeutic burden” as used herein pertain to the control of plasma glucose concentrations in a diabetic patient. Existing insulin-based treatments for type 1 diabetes place the therapeutic burden upon insulin to achieve control of plasma glucose concentrations, at the risk of plasma glucose falling too low and an onset of hypoglycemia. Alternatively, according to methods disclosed herein, whereby insulin and an amylin analog are separately co-administered to a patient with type 1 diabetes, and whereby a concentration of amylin analog is administered at a therapeutically effective dose that is at or greater than the ED70 dose of the amylin agonist, the therapeutic burden shifts to the glucose dependent hormone amylin. The methods disclosed herein reduce the therapeutic burden of co-administered insulin, thus permitting relatively lower and safer doses of insulin, putting the patient at diminished risk of hypoglycemia.

The terms “drug,” “therapeutic agent,” and “beneficial agent” are used interchangeably to refer to any therapeutically active substance that is delivered to a subject to produce a desired beneficial effect. In one embodiment of the present disclosure, the drug is a polypeptide. In another embodiment of the present disclosure, the drug is a small molecule, for example, hormones such as androgens or estrogens. The devices and methods of the present disclosure are well suited for the delivery of proteins, small molecules and combinations thereof.

The terms “peptide,” “polypeptide,” and “protein” are used interchangeably herein and typically refer to a molecule comprising a chain of two or more amino acids (e.g., most typically L-amino acids, but also including, e.g., D-amino acids, modified amino acids, amino acid analogs, and amino acid mimetics).

The terminal amino acid at one end of the peptide chain typically has a free amino group (i.e., the amino terminus). The terminal amino acid at the other end of the chain typically has a free carboxyl group (i.e., the carboxy terminus). Typically, the amino acids making up a peptide are numbered in order, starting at the amino terminus and increasing in the direction of the carboxy terminus of the peptide.

The phrase “amino acid residue” as used herein refers to an amino acid that is incorporated into a peptide by an amide bond or an amide bond mimetic.

As used herein, the term “HbA1c” refers to glycated hemoglobin. It develops when hemoglobin, a protein within red blood cells that carries oxygen throughout your body, joins with glucose in the blood, becoming “glycated.” By measuring glycated hemoglobin (HbA1c), clinicians are able to get an overall picture of what our average blood sugar levels have been over a period of weeks/months. For people with diabetes this is important as the higher the HbA1c, the greater the risk of developing diabetes-related complications. HbA1c is also referred to as hemoglobin A1c or simply A1c.

The term “insulinotropic” as used herein typically refers to the ability of a compound. e.g., a peptide, to stimulate or affect the production and/or activity of insulin (e.g., an insulinotropic hormone). Such compounds typically stimulate or otherwise affect the secretion or biosynthesis of insulin in a subject. Thus, an “insulinotropic peptide” is an amino acid-containing molecule capable of stimulating or otherwise affecting secretion or biosynthesis of insulin.

The term “insulinotropic peptide” as used herein includes, but is not limited to, glucagon-like peptide 1 (GLP-1), as well as derivatives and analogues thereof, GLP-1 receptor agonists, such as exenatide.

The phrase “incretin mimetics” as used herein includes but is not limited to GLP-1 peptide, GLP-1 receptor agonists, peptide derivatives of GLP-1, and peptide analogs of GLP-1. Incretin mimetics are also referred to herein as “insulinotropic peptides.”

The term “GLP-1” refers to a polypeptide that is produced by the L-cell located mainly in the ileum and colon, and to a lesser extent by L-cells in the duodenum and jejunum. GLP-1 is a regulatory peptide that binds to the extracellular region of the GLP-1 receptor (GLP-1R), a G-coupled protein receptor on β cell and via adenyl cyclase activity and production of cAMP stimulates the insulin response to the nutrients that are absorbed from the gut [Baggio 2007, “Biology of incretins: GLP-1 and GIP,” Gastroenterology, vol. 132(6):2131-57; Holst 2008, “The incretin system and its role in type 2 diabetes mellitus,” Mol Cell Endocrinology, vol. 297(1-2):127-36]. The effects of GLP-1R agonism are multiple. GLP-1 maintains glucose homeostasis by enhancing endogenous glucose dependent insulin secretion, rendering the p cells glucose competent and sensitive to GLP-1, suppressing glucagon release, restoring first and second phase insulin secretion, slowing gastric emptying, decreasing food intake, and increasing satiety [Hoist 2008 Mol. Cell Endocrinology; Kjems 2003 “The influence of GLP-1 on glucose-stimulated insulin secretion: effects on beta-cell sensitivity in type 2 and nondiabetic subjects.” Diabetes, vol. 52(2): 380-86; Hoist 2013 “Incretin hormones and the satiation signal,” Int J Obes (Lond), vol. 37(9):1161-69; Seufert 2014, “The extra-pancreatic effects of GLP-1 receptor agonists: a focus on the cardiovascular, gastrointestinal and central nervous systems,” Diabetes Obes Metab, vol. 16(8): 673-88]. The risk of hypoglycemia is minimal given the mode of action of GLP-1. One example of a GLP-1 receptor agonist is Victoza+ (Novo Nordisk A/S, Bagsvaerd D K) (liraglutide; U.S. Pat. Nos. 6,268,343, 6,458,924, and 7,235,627). Once-daily injectable Victoza® (liraglutide) is commercially available in the United States, Europe, and Japan. Another example of a GLP-1 receptor agonist is Ozempic® or Rybelsus® (Novo Nordisk A/S, Bagsvaerd D K) (semaglutide, injectable and orally administered formulations, respectively). A further example of a GLP-1 receptor agonist is exenatide. For ease of reference herein, the family of GLP-1 receptor agonists, GLP-1 peptides, GLP-1 peptide derivatives and GLP-1 peptide analogs having insulinotropic activity is referred to collectively as “GLP-1.”

As used herein, the term “amylin” refers to a human peptide hormone of 37 amino acids, which is co-secreted with insulin from β-cells of the pancreas. Human amylin has the following amino acid sequence (three letter code): Lys-Cys-Asn-Thr-Ala-Thr-Cys-Ala-Thr-Gin-Arg-Leu-Ala-Asn-Phe-Leu-Val-His-Ser-Ser-Asn-Asn-Phe-Gly-Ala-lie-Leu-Ser-Ser-Thr-Asn-Val-Gly-Ser-Asn-Thr-Tyr (SEQ ID NO:5). Thus, the structural formula is Lys-Cys-Asn-Thr-Ala-Thr-Cys-Ala-Thr-Gin-Arg-Leu-Ala-Asn-Phe-Leu-Val-His-Ser-Ser-Asn-Asn-Phe-Gly-Ala-Ile-Leu-Ser-Ser-Thr-Asn-Val-Gly-Ser-Asn-Thr-Tyr-NH₂ (SEQ ID NO: 5) with a disulfide bridge between the two Cys residues and an amide group attached to the C-terminal amino acid via a peptide bond. The term “amylin” also includes variants of amylin as present in, and in isolatable form, other mammalian species. With respect to a naturally occurring amylin compound, the term includes such a compound in an isolated, purified, or other form that is otherwise not found in nature.

As used herein, the term “agonist” is used in the broadest sense and includes any molecule that mimics a biological activity of a native polypeptide disclosed herein. Suitable agonist molecules specifically include agonist antibodies or antibody fragments, fragments or amino acid sequence variants of native polypeptides, peptides, small organic molecules, etc. Methods for identifying agonists of a native polypeptide may comprise contacting a native polypeptide with a candidate agonist molecule and measuring a detectable change in one or more biological activities normally associated with the native polypeptide.

As used herein, the terms “amylin analog” and “amylin receptor agonist” are used interchangeably herein and refer to a compound that mimics one or more effects (or activity) of amylin in vitro or in vivo. The effects of amylin include the ability to directly or indirectly interact or bind with one or more receptors that are activated or deactivated by amylin. For example, amylin agonists as used herein are compounds having at least 60, 65, 70, 75, 80, 85, 90, 95, or 99% amino acid sequence identity to SEQ ID NO: 5 and having amylin activity. Amylin agonists include human amylin, mammalian amylins, vertebrate amylins, rodent amylins, amylin derivatives described in U.S. Pat. No. 5,656,590, CGRP and analogs, avian calcitonins, teleost calcitonins including salmon and eel calcitonins, calcitonins as described in U.S. Pat. No. 5,321,008, davlintide, pramlintide and other amylin analog compositions described in U.S. Pat. No. 7,271,238, compositions described in U.S. Pat. No. 6,610,824, compositions claimed in U.S. Pat. No. 8,497,347, compositions claimed in US patent application 2012/0046224, U.S. Pat. Nos. 9,023,789, 8,486,890, 8,575,091, in U.S. Pat. Nos. 8,895,504, 8,114,958, and US patent application publications 2012/0046224, 2011/0105394, 2011/0152183, 2010/0222269 and 2009/0099085.

As used herein, the terms “analog” or “analogue” or “agonist analog” of amylin refers to a compound that is similar in structure (e.g., derived from the primary amino acid sequence of amylin by substituting one or more natural or unnatural amino acids or peptidomimetics) to amylin and mimics an effect of amylin in vitro or in vivo. As used herein, the term “amylin agonist” refers to an amylin analog

As used herein, an amylin analog comprises, for example, amylin having insertions, deletions, and/or substitutions in at least one or more amino acid positions of SEQ ID NO: 5. The number of amino acid insertions, deletions, or substitutions may be at least 1, 2, 3, 4, 5, 6, or 10. Insertions or substitutions may be with other natural or unnatural amino acids, synthetic amino acids, peptidomimetics, or other chemical compounds. Amylin agonists include human amylin, vertebrate amylins, amylin derivatives described in U.S. Pat. No. 5,656,590, calcitonin gene related peptide (CGRP) and analogs, avian calcitonins, teleost calcitonins including salmon and eel calcitonins, calcitonins as described, e.g., in U.S. Pat. Nos. 5,321,008, 8,486,890, pramlintide, Symlin®, and other amylin analog compositions described in, e.g., U.S. Pat. Nos. 7,271,238, 5,321,008, 5,367,052, compositions claimed in, e.g., U.S. Pat. No. 8,497,347, compositions claimed in, e.g., U.S. patent application Ser. No. 12/601,884.

As used herein, a “derivative” of amylin refers to an amylin which is chemically modified. e.g. by introducing a side chain in one or more positions of the amylin backbone or by oxidizing or reducing groups of the amino acid residues in the amylin or by converting a free carboxylic group to an ester group or to an amide group. Other derivatives are obtained by acylating a free amino group or a hydroxy group.

As described in greater detail below, in some embodiments, the amylin analog polypeptides disclosed herein are provided in methods for treatment of type 1 diabetes, as an adjunct to treatment with insulin.

The term “insulin,” as used herein, refers to human insulin or any insulin analogs. Exemplary non-limiting insulin analogs include those listed in Table 1:

TABLE 1
Exemplary insulin analogs

| | | | | |
|---|---|---|---|---|
| | | | | Role in Blood Sugar |
| Type of Insulin & Brand Name | Onset | Peak | Duration | Management |

“Ultra Fast” Rapid-Acting

| | | | | | |
|---|---|---|---|---|---|
| Fiasp ® (aspart) | about 5 mins | 1-3 | hours | 3-5 | hours |
| | sooner than | | | | |
| | Rapid Acting | | | | |
| | Insulins | | | | |

Rapid-Acting (enter the bloodstream within minutes, for injection within 5 to 10 minutes of eating; peak
action period of 60-120 minutes, and clears after about four hours; used in continuous subcutaneous
insulin infusion)

| | | | | | | | |
|---|---|---|---|---|---|---|---|
| Lilly's Humalog ® (lispro) | 15-30 | min. | 30-90 | min | 3-5 | hours | Rapid-acting insulins |
| Novo's Novolog ® (aspart) | 10-20 | min. | 40-50 | min. | 3-5 | hours | cover insulin needs |
| Sanofi's Apidra ® (glulisine) | 20-30 | min. | 30-90 | min. | 1-2½ | hours | for meals eaten at |
| Sanofi's Admelog ® (lispro) | 15-30 | min. | 30-90 | min. | 3-5 | hours | the same time as the |
| | | | | | | | injection. This type of |
| | | | | | | | insulin is often used |
| | | | | | | | with longer-acting |
| | | | | | | | insulin. |

Short-Acting

| | | | | | | |
|---|---|---|---|---|---|---|
| Novo's Novolin ® | 30 min.-1 hour | 2-5 | hours | 5-8 | hours | Short-acting insulins |
| (recombinant insulin) | | | | | | cover insulin needs |
| velosulin (human insulin | 30 min.-1 hour | 1-2 | hours | 2-3 | hours | for meals eaten within |
| for use in an insulin pump) | | | | | | 30-60 minutes. |

Intermediate-Acting

| | | | | | | | |
|---|---|---|---|---|---|---|---|
| neutral protamine | 1-2 | hours | 4-12 | hours | 18-24 | hours | Intermediate-acting |
| hagedorn (NPH) insulin | | | | | | | insulin covers insulin |
| | | | | | | | needs for about half |
| | | | | | | | the day or overnight. |
| | | | | | | | This type of insulin |
| | | | | | | | is often combined with |
| | | | | | | | a rapid- or short-acting |
| | | | | | | | type. |

Long-Acting (suitable for background or basal insulin replacement)

| | | | | | | |
|---|---|---|---|---|---|---|
| Lilly's Basaglar ® (100 | 1-1½ | hours | No peak time. | 20-24 | hours | Long-acting insulins |
| units/mL); | | | Delivered at a | | | cover insulin needs |
| Sanofi's Lantus ® (100 | | | steady level. | | | for about one full day. |
| units/mL) & Toujeo ® (300 | | | | | | This type is often |
| units/mL) | | | | | | combined, when needed, |
| (insulin glargine) | | | | | | with rapid- or |
| Usually injected once daily, | | | | | | short-acting insulin. |
| but may be given twice daily. | | | | | | |
| Insulin glargine aggregates | | | | | | |
| into clusters when injected. | | | | | | |
| Individual insulin units detach | | | | | | |
| from the cluster, for absorption | | | | | | |
| into the blood stream. Slow | | | | | | |
| break-up of these clusters | | | | | | |
| contribute to insulin glargine's | | | | | | |
| long action. | | | | | | |

| | | | | | |
|---|---|---|---|---|---|
| Novo's Levemir ® (insulin | 1-2 | hours | 6-8 | hours | Up to 24 hours |
| detemir) | | | | | |
| Suitable for twice daily | | | | | |
| injection. | | | | | |
| Insulin detemir is absorbed | | | | | |
| into the blood stream, binds | | | | | |
| human serum albumin (HSA), | | | | | |
| and provides relatively steady | | | | | |
| concentrations, over 12 to 24 | | | | | |
| hours, of low levels of | | | | | |
| unbound or “free” detemir. | | | | | |

| | | | | | |
|---|---|---|---|---|---|
| Novo's Tresiba ® | 30-90 | min. | No peak time | 42 | hours *** |
| (insulin degludec) | | | | | |

Pre-Mixed* Insulins

| Lilly's Humulin ® 70/30 | 30 | min. | 2-4 | hours | 14-24 | hours | These products are |
|---|---|---|---|---|---|---|---|
| Novo's Novolin ® 70/30 | 30 | min. | 2-12 | hours | Up to 24 hours | | generally taken |
| Novo's Novolog ® 70/30 | 10-20 | min. | 1-4 | hours | Up to 24 hours | | two or three times |
| Lilly's Humulin ® 50/50 | 30 | min. | 2-5 | hours | 18-24 | hours | a day before |

| | | | | | | |
|---|---|---|---|---|---|---|
| Lilly's Humalog ® mix 75/25 | 15 | min. | 30 min.-2½ hours | 16-20 | hours | mealtime. |

*Premixed insulins combine specific amounts of intermediate-acting and short-acting insulin in one unit or
insulin pen. (The numbers following the brand name indicate the percentage of each type of insulin.)
Insulin/GLP-1 receptor agonist combinations

| | | | | | |
|---|---|---|---|---|---|
| Novo's Xultophy ®) | 30-90 | min. | No peak time | 42 | hours |
| (insulin degludec 100 | | | | | |
| units/mL & liraglutide 3.6 | | | | | |
| mg/mL) | | | | | |
| Sanofi's Soliqua ® | 1-1½ | hours | No peak time | 20-24 | hours |
| (insulin glargine 100 units/mL | | | | | |
| & lixisenatide 33 mcg/mL) | | | | | |

The term “meal-time insulin” as used herein refers to a fast-acting insulin formulation that reaches peak blood concentration in approximately 45-90 minutes and peak activity approximately 1 to 3 hours after administration and is administered at or around mealtime.

Those of ordinary skill in this art will recognize that the above terms “insulin” and “amylin” can be read broadly to include any polypeptide or other chemical class having the above described desired biological activity, in vitro or in vivo, which stimulates or suppresses, respectively, glucose incorporation into glycogen in any of many test systems, including, rat soleus muscle. In addition, such persons recognize that the polypeptide may be provided in a form which does not significantly affect the desired biological activity of the polypeptide. For example, as described in U.S. Pat. No. 5,124,314 or U.S. Pat. No. 5,641,744, the amylin may be prepared in a soluble form.

The term “glucose regulating peptide” as used herein refers to any peptide that controls glucose metabolism, including serum levels, glucogenesis, glucose breakdown, glucose uptake, glucose storage, and glucose release. Representative glucose regulating peptides comprise amylin and insulin, and their analogs, disclosed herein.

The term “normoglycemia” (or “euglycemia”) as used herein means a normal concentration of glucose in the blood or plasma of a patient. As used herein, normoglycemia may refer to a range of blood glucose concentrations (normoglycemic range) found in healthy populations. Those skilled in the art will recognize variations in the normoglycemic range depending on the individual or patient population in question.

As used herein an “effective” amount or a “therapeutically effective amount” of a peptide refers to a nontoxic but sufficient amount of the peptide to provide the desired effect. For example, one desired effect would be the prevention or treatment of hypoglycemia, as measured, for example, by an increase in blood glucose level. An alternative desired effect for the peptides of the present disclosure would include treating hyperglycemia, e.g., as measured by a change in blood glucose level closer to normal, or inducing weight loss/preventing weight gain, e.g., as measured by reduction in body weight, or preventing or reducing an increase in body weight, or normalizing body fat distribution. The amount that is “effective” will vary from subject to subject, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

The term “implantable delivery device” as used herein typically refers to a delivery device that is fully implanted beneath the surface of a subject's skin to affect administration of a drug.

Representative implantable delivery devices include Hydron® Implant Technology, from Valera Pharmaceuticals. Inc.; NanoGATE™ implant, from iMEDD Inc.; MIP implantable pump or DebioStar™ drug delivery technology, from Debiotech S.A.; Prozor™, Nanopor™ or Delos Pump™, from Delpor Inc.; or an implantable osmotic delivery device, e.g., ITCA-0650, from Intarcia Therapeutics, Inc.

The terms “osmotic delivery device” and “implantable osmotic delivery device” are used interchangeably herein and typically refer to a device used for delivery of a drug to a subject, wherein the device comprises, for example, a reservoir (made, e.g., from a titanium alloy) having a lumen that contains a suspension formulation comprising a drug and an osmotic agent formulation. A piston assembly positioned in the lumen isolates the suspension formulation from the osmotic agent formulation. A semi-permeable membrane is positioned at a first distal end of the reservoir adjacent the osmotic agent formulation and a diffusion moderator (which defines a delivery orifice through which the suspension formulation exits the device) is positioned at a second distal end of the reservoir adjacent the suspension formulation. Typically, the osmotic delivery device is implanted within the subject, for example, subdermally or subcutaneously (e.g., in the inside, outside, or back of the upper arm and in the abdominal area). An exemplary osmotic delivery device is the DUROS® (ALZA Corporation, Mountain View, Calif.) delivery device. Examples of terms synonymous to “osmotic delivery device” include but are not limited to “osmotic drug delivery device”, “osmotic drug delivery system”, “osmotic device”, “osmotic delivery device”, “osmotic delivery system”, “osmotic pump”, “implantable drug delivery device”, “drug delivery system”, “drug delivery device”, “implantable osmotic pump”, “implantable drug delivery system”, and “implantable delivery system”. Other terms for “osmotic delivery device” are known in the art.

Typically, for an osmotic delivery system, the volume of the chamber comprising the drug formulation is between about 100 μl to about 1000 μl, more preferably between about 140 μl and about 200 μl. In one embodiment, the volume of the chamber comprising the drug formulation is about 150 μl.

The term “non-implantable delivery device” as used herein typically refers to a delivery device, including a “non-implantable miniaturized patch pump,” having certain components that are not implanted beneath the surface of a subject's skin to affect administration of a drug.

Representative non-implantable delivery devices (e.g., patch pumps) include Omnipod®, from Insulet Corp.; Solo™, from Medingo; Finesse™, from Calibra Medical Inc.; Cellnovo pump, from Cellnovo Ltd.; CeQur™ device, from CeQur Ltd.; Freehand™, from MedSolve Technologies, Inc.; Medipacs pump, from Medipacs, Inc.; Medtronic pump and MiniMed Paradigm, from Medtronic, Inc.; Nanopump™, from Debiotech S.A. and STMicroelectronics; NiliPatch pump, from NiliMEDIX Ltd.; PassPort®, from Altea Therapeutics Corp.; SteadyMed patch pump, from SteadyMed Ltd.; V-Go™, from Valeritas, Inc.; Finesse, from LifeScan; JewelPUMP™, from Debiotech S.A.; SmartDose Electronic Patch Injector, from West Pharmaceutical Services, Inc.; SenseFlex FD (disposable) or SD (semi-disposable), from Sensile Medical A.G.; Asante Snap, from Bigfoot Biomedical; PicoSulin device, from PicoSulin; and Animas® OneTouch Ping Pump, from Animas Corp.

In some embodiments, the non-implantable miniaturized patch pump is, e.g., JewelPUMP™ (Debiotech S.A.), placed on the surface of the skin. Dosing of the JewelPUMP™ device is adjustable and programmable. The JewelPUMP™ is based on a microelectromechanical system (MEMS) integrated and ultra-precise disposable pump-chip technology. The JewelPUMP™ is a miniaturized patch-pump with a disposable unit having payload for administration of compound. The disposable unit is filled once with compound and discarded after use, while the controller unit (including the electronics) can be used for 2 years with multiple disposable units. In some embodiments, the JewelPUMP™ is detachable, watertight for bathing and swimming, includes direct access bolus buttons and a discreet vibration & audio alarm on the patch-pump. In some embodiments, the JewelPIUMP™ is remotely controlled.

The term “continuous delivery” as used herein typically refers to a substantially continuous release of drug from an osmotic delivery device and into tissues near the implantation site, e.g., subdermal and subcutaneous tissues. For example, an osmotic delivery device releases drug essentially at a predetermined rate based on the principle of osmosis. Extracellular fluid enters the osmotic delivery device through the semi-permeable membrane directly into the osmotic engine that expands to drive the piston at a slow and consistent rate of travel. Movement of the piston forces the drug formulation to be released through the orifice of the diffusion moderator. Thus release of the drug from the osmotic delivery device is at a slow, controlled, consistent rate.

The term “substantial steady-state delivery” as used herein typically refers to delivery of a drug at or near a target concentration over a defined period of time, wherein the amount of the drug being delivered from an osmotic delivery device is substantially zero-order delivery. Substantial zero-order delivery of an active agent (e.g., a disclosed amylin analog polypeptide) means that the rate of drug delivered is constant and is independent of the drug available in the delivery system; for example, for zero-order delivery, if the rate of drug delivered is graphed against time and a line is fitted to the data the line has a slope of approximately zero, as determined by standard methods (e.g., linear regression).

The phrase “drug half-life” as used herein refers how long it takes a drug to be eliminated from blood plasma by one half of its concentration. A drug's half-life is usually measured by monitoring how a drug degrades when it is administered via injection or intravenously. A drug is usually detected using, for example, a radioimmunoassay (RIA), a chromatographic method, an electrochemiluminescent (ECL) assay, an enzyme linked immunosorbent assay (ELISA) or an immunoenzymatic sandwich assay (IEMA).

The terms “μg” and “mcg” and “ug” are understood to mean “micrograms”. Similarly, the terms “μl” and “uL” are understood to mean “microliter”, and the terms “μM” and “uM” are understood to mean “micromolar”.

The term “serum” is meant to mean any blood product from which a substance can be detected. Thus, the term serum includes at least whole blood, serum, and plasma. For example, “an amount of [a substance] in a subject's serum” would cover “an amount of [the substance] in a subject's plasma”.

Baseline is defined as the last assessment on or before the day of the initial placement of an osmotic delivery device (containing drug or placebo).

### Endogenous Amylin, Related Peptides and Amylin Receptors

Human amylin, a 37-residue polypeptide hormone, is co-secreted with insulin from the pancreatic β-cells. Loss of β-cell function that occurs early in type 1 diabetics and can occur late in type 2 diabetics leads to deficiencies in the secretion of insulin and amylin. Amylin is believed to play a role in glycemic regulation by slowing gastric emptying and promoting satiety, thereby preventing post-prandial spikes in blood glucose levels. The overall effect is to slow the rate of appearance of glucose in the blood after eating.

Amylin's amino acid sequence is most closely related to that of calcitonin gene-related peptide (CGRP). CGRP also shares a similarly positioned disulfide bond and an amidated C-terminus. This is also the case for calcitonin, adrenomedullin, and adrenomedullin 2. Together, these peptides form a small family, united by these characteristic features. Consequently, there is a degree of overlap in binding the cognate receptors for each peptide and pharmacological activity.

The peptides typically designated as calcitonin (CT) peptide family members include: calcitonin gene-related peptide (CGRP), calcitonin (CT), amylin (AMY), adrenomedullin 1, and adrenomedullin 2/intermedin (ADM1, ADM2 respectively). Two G protein-coupled receptor proteins (calcitonin receptor; CTR, and calcitonin-receptor-like receptor; CALCRL) and three receptor activity-modifying proteins, (RAMP1, RAMP2, RAMP3) make up the pharmacologically distinct receptors for the entire peptide family (CTR, AMY1, AMY2, AMY3, CGRPR, AM1, AM2). There appear to be at least five distinct receptors to which amylin binds with significant affinity (AMY1, AMY2, AMY3, CTR, CGRPR). CTR dimerizes with RAMPs 1, 2, or 3 to reconstitute the AMY1, AMY2, or AMY3 receptors with pharmacology selective for amylin over calcitonin. In the absence of a RAMP, CTR pharmacology becomes calcitonin selective versus amylin. CALCRL dimerized with RAMP1 generates CGRPR with high affinity for CGRP and reduced affinities for all other peptide family members including amylin. CALCRL and RAMP2, or RAMP3, reconstitute the pharmacology of AM1, and AM2 respectively with very low to no affinity for amylin.

Amylin analog polypeptides, having binding affinity to amylin receptor complexes, have been developed. Pramlintide, for example, was developed by Amylin Pharmaceuticals, and approved by the U.S. Food and Drug Administration (FDA), as a synthetic analogue of human amylin for the treatment of types 1 and 2 diabetics, who use meal-time insulin but cannot achieve desired glycemic control despite optimal insulin therapy. Pramlintide is an amylinomimetic agent that is at least as potent as human amylin. It is also a 37-amino-acid polypeptide and differs in amino acid sequence from human amylin by replacement of amino acids with proline at positions 25 (alanine), 28 (serine), and 29 (serine). As a result of these substitutions, pramlintide is soluble, non-adhesive, and nonaggregating, thereby overcoming a number of the physicochemical liabilities of native human amylin. The half-life of pramlintide is approximately 48 minutes in humans, longer than that of native human amylin (about 13 minutes). Pramlintide requires frequent and inconvenient administration.

For treatment of type 1 diabetics, pramlintide is administered up to four times per day, via subcutaneous injection in the thigh or abdomen before meals, as an adjunct to insulin therapy administered after meals. Pramlintide cannot be mixed with insulin; separate syringes are used. Pramlintide is administered with or prior to each meal or snack that consists of at least 250 calories or 30 g of carbohydrate. The typical starting dose for type 1 diabetics is 15 μg subcutaneous pramlintide before each meal, with subsequent titration to a target dose of 60 μg before each meal. Reported side effects of pramlintide include nausea and vomiting. Adverse reactions, particularly for type 1 diabetics, can include severe hypoglycemia. Consequently, dosage of meal-time insulin is reduced for diabetic patients who initiate administration of pramlintide.

For treatment of type 2 diabetics, pramlintide is administered via subcutaneous injection at a recommended starting dose of 60 μg, with a target maintenance dose of 120 μg before each meal.

Davalintide (AC2307) is another analog of human amylin. Davalintide is an investigational compound with a half-life of about 26 minutes. Like pramlintide, davalintide would likewise require frequent administration via injection.

In some embodiments, the amylin analog is selected from the group consisting of those disclosed in U.S. patent application Ser. No. 16/598,915, the entire contents of which is incorporated herein by reference. In some embodiments, the amylin analog comprises an amino acid sequence selected from the group consisting of those in Table 2:

TABLE 2
Exemplary amylin analog polypeptides

| Compound No. | Sequence | SEQ ID NO |
|---|---|---|
| A1 | SC*NTSTC*ATQRLANEk*((γGlu)₂-CO(CH₂)₁₄CH₃)HKSSNNFGPILPPTKVGSETY-NH₂ | SEQ ID NO: 1 |
| A2 | K*((γGlu)₂(CO(CH₂)₁₈CO₂H))C*NTSTC*ATQRLANELHKSSNNFGPILPPTKVGSETY-(NH₂) | SEQ ID NO: 2 |
| A3 | K*((γGlu)₂(CO(CH₂)₁₆CO₂H))C*NTSTC*ATQRLANELHKSSNNFGPILPPTKVGSETY-(NH₂) | SEQ ID NO: 3 |
| A4 | K*(γGlu-CO(CH₂)₁₆CO₂H)C*NTSTC*ATSRLANFLQKSSNNFGPILPPTKVGSETY-NH₂ | SEQ ID NO: 4 |

Note:
the two cysteine residues denoted C* are bound by a disulfide bridge;
k represents D-lysine

In some embodiments, an isolated polypeptide of the disclosure comprises an amino acid sequence: SC*NTSTC*ATQRLANEk*((γGlu)₂-CO(CH₂)₁₄CH₃)HKSSNNFGPILPPTKVGSE TY-NH₂ (SEQ ID NO: 1), which is also referred to herein as Compound A1.

In some embodiments, an isolated polypeptide of the disclosure comprises an amino acid sequence:K*((γGlu)₂(CO(CH₂)₁₈CO₂H))C*NTSTC*ATQRLANELHKSSNNFGPILPPTKV GSETY-(NH₂) (SEQ ID NO: 2), which is also referred to herein as Compound A2.

In some embodiments, the amylin analog comprises an amino acid sequence: K*((γGlu)₂(CO(CH₂)₁₈CO₂H))C*NTSTC*ATQRLANELHKSSNNFGPILPPTKVGSETY-(NH₂) (SEQ ID NO: 3), which is also referred to herein as Compound A3.

In some embodiments, the amylin analog comprises an amino acid sequence: K*(γGlu-CO(CH₂)₁₆CO₂H)C*NTSTC*ATSRLANFLQKSSNNFGPILPPTKVGSETY-NH₂ (SEQ ID NO: 4), which is also referred to herein as Compound A4.

Certain disclosed amylin analog polypeptides were developed for administration via weekly or monthly injections. Certain disclosed amylin analog polypeptides were developed for administration via implantation of a delivery device comprising the amylin analog polypeptide, where the delivery device comprises a dose of the amylin analog polypeptide of up to 3 months, 6 months, 9 months, one year, 18 months or two years.

### Description of Exemplary Embodiments

In certain embodiments, this disclosure provides methods of (i) continuous administration of the amylin analog; and (ii) administration of the amylin analog at a high therapeutically effective dose relative to known amylin treatment regimens.

In some embodiments, continuous administration of the amylin analog is achieved via an implantable (e.g., osmotic) or non-implantable (external infusion pump) drug delivery device. Both short-acting amylin analogs (e.g., pramlintide) or long acting amylin analogs (e.g., compound A2, described herein) can be administered to the patient via an implantable (e.g., osmotic) or non-implantable (external infusion pump) drug delivery device to achieve continuous administration. Further, continuous administration of a long-acting amylin analog (e.g., compound A2) can also be achieved in the patient by administration via infrequent (e.g., once weekly) injections.

In some embodiments, the amylin analog is provided at a high therapeutically effective dose relative to known amylin treatment regimens. In certain embodiments, methods are provided of administering to a patient an amylin analog at a high therapeutically effective dose of at least 5 μg per kilogram per day. In certain embodiments, methods are provided of administering to a patient an amylin analog at a high therapeutically effective dose of at least: 6 μg per kilogram per day, 7 μg per kilogram per day, 8 μg per kilogram per day, 9 μg per kilogram per day, 10 μg per kilogram per day, 12 μg per kilogram per day, 14 μg per kilogram per day, 16 μg per kilogram per day, 18 μg per kilogram per day, 20 μg per kilogram per day, 25 μg per kilogram per day, 30 μg per kilogram per day, 35 μg per kilogram per day, 40 μg per kilogram per day, 45 μg per kilogram per day, 50 sg per kilogram per day, 75 μg per kilogram per day or 100 μg per kilogram per day.

In certain other embodiments, methods are provided of administering to a patient an amylin analog at a high therapeutically effective dose that is at or greater than the ED70 dose of the amylin agonist. In certain other embodiments, methods are provided of administering to a patient an amylin analog at a therapeutically effective dose that is at or greater than the ED75, ED80, ED85, ED90 or ED95 dose of the amylin agonist.

One aspect of the disclosure provides a method of treating diabetes mellitus, comprising administering to a patient in need thereof an amylin analog at a therapeutically effective dose that is at or greater than the ED70 dose of the amylin agonist.

One aspect of the disclosure provides a method of improving and stabilizing or normalizing glucose levels in a patient in need thereof, comprising administering to a patient in need thereof an amylin analog at a therapeutically effective dose that is at or greater than the ED70 dose of the amylin agonist. In some embodiments, the amylin analog is an agent that activates a heterodimeric receptor constituted from a calcitonin receptor and an amylin 3 receptor. In some embodiments, the amylin 3 receptor is a human amylin 3 receptor.

One aspect of the disclosure provides a method of maintaining normoglycemia in a patient in need thereof, comprising administering to a patient in need thereof an amylin analog at a therapeutically effective dose that is at or greater than the ED70 dose of the amylin agonist. In some embodiments, the amylin analog is an agent that activates a heterodimeric receptor constituted from a calcitonin receptor and an amylin 3 receptor. In some embodiments, the amylin 3 receptor is a human amylin 3 receptor.

In some embodiments, the at least 70% activation of amylin receptors is achieved using an in vitro system. In some embodiments, the at least 70% amylin activation is detected using an amylin activity assay. In some embodiments, the at least 70% amylin activation is detected using an amylin activity assay as described in U.S. Pat. No. 6,048,514, the contents of which are hereby incorporated by reference in their entirety. In some embodiments, the amylin activity assay comprises (i) bringing together a test sample and a test system, said test sample comprising one or more test compounds, and said test system comprising an in vivo biological model, said in vivo model being characterized in that it exhibits elevated lactate and elevated glucose in response to the introduction to said model of amylin or an amylin agonist; (ii) determining the presence or amount of a rise in lactate and the presence or amount of a rise in glucose in said test system. (iii) determining whether a peak in elevated lactate preceded a peak in elevated glucose; and (iv) identifying those test compounds which resulted in a peak in elevated lactate which preceded a peak in elevated glucose in the in vivo biological model in which at least one test compound in the test sample brought together with the test system results in a peak in elevated lactate which precedes a peak in elevated glucose.

Embodiments herein provide continuous administration of an amylin analog according to methods know in the art. For example, the amylin analog may be provided by either an implantable drug delivery device such as an osmotic drug delivery device, capable of continuous amylin administration. Alternatively, the amylin analog may be provided by a non-implantable drug delivery device. The amylin analog may be provided by infusion devices, such as pumps, that continuously administer amylin to a patient. In some embodiments, continuous infusion is provided by an external device capable of subcutaneous, intra-muscular, intra-peritoneal, intra-abdominal, intravenous, or any suitable manner of administration.

Insulin therapy for the treatment of type 1 diabetes requires high patient adherence that requires numerous self-injections. Insulin therapy is prone to significant fluctuations in serum glucose concentrations which can drift outside an intended healthy range of approximately 70 mg/dL to 180 mg/dL. As used herein, the term “time-in-range” refers to the fraction of time (e.g., per day, per week, per month, et.) in which a type 1 diabetic patient maintains, under therapy, serum glucose concentrations of approximately 70 mg/dL to 180 mg/dL. Correspondingly, the term “time-out-of-range” refers to a length of time (e.g., per day, per week, per month, et.) in which a type 1 diabetic patient fails to maintain, under therapy, serum glucose concentrations of approximately 70 mg/dL to 180 mg/dL. Hyperglycemia occurs when the patient's serum glucose concentrations exceeds 180 mg/dL Hyperglycemia is an unhealthy condition that contributes to cardiovascular and microvascular issues but does not generally present an immediate threat to a patient's wellbeing. Hypoglycemia, by contract, can present an immediate threat that can cause a patient to become cognitively impaired, become unconscious, or go into a coma.

The presently described methods present a significant opportunity to improve the health and quality of life of type 1 diabetes patients. Advantages of the presently described methods include significantly simplified treatment regimens, reduced need for glucose monitoring, reduced insulin usage and administration, reduced treatment burden, improved quality of life, reduced risk of hypoglycemia, avoidance of weight gain associated with insulin, reduced HbA1c and increased time-in-range.

In some embodiments, a method is provided of treating type 1 diabetes in a human subject, comprising administering to the subject a pharmaceutical composition comprising an amylin analog at a therapeutically effective dose:
(i) of at least 5 μg per kilogram of the subject per day; or
(ii) that is at or greater than the ED75 dose of the amylin analog.

Also disclosed herein is a pharmaceutical composition comprising an amylin analog for use in the treatment of type 1 diabetes in a human subject, the use comprising administering to the subject a therapeutically effective dose of the amylin analog that is:
(i) at least 5 μg per kilogram of the subject per day; or
(ii) at or greater than the ED75 dose of the amylin analog.

In some embodiments, the method further comprises continuously maintaining a concentration of the amylin analog in the subject that is at or greater than the EC75 dose of the amylin agonist.

In some embodiments, the method comprises continuous administration of the amylin analog. In some embodiments, the method comprises continuous administration of the amylin analog via an implantable drug delivery device. In some embodiments, the implantable drug delivery device in an osmotic drug delivery device. In some embodiments, the method comprises continuous administration of the amylin analog via a non-implantable drug delivery device. In some embodiments, the method comprises continuous administration of the amylin analog via injection twice per week, once weekly injection, or injection less frequently than once per week, such as injection once per month or injection four times per year. In some embodiments, the amylin analog is pramlintide. In some embodiments, the amylin analog is compound A2 (SEQ ID NO:2). In some embodiments, the method further comprises separate administration of an insulin.

### Uses, Formulation and Administration

Compositions

In some embodiments, an amylin analog polypeptide of the disclosure is co-formulated in combination with insulin or an insulin derivative. In some embodiments, an amylin analog polypeptide of the disclosure is co-formulated in combination with a long-acting basal insulin or long-acting basal insulin derivative.

In embodiments of this disclosure, compositions are provided comprising amylin, an amylin analog, insulin, or an insulin analog, or combinations thereof, for treating a patient suffering from a condition where insulin or amylin treatment is indicated. In certain embodiments, an amylin analog is provided, alone or in conjunction with an insulin, suitable for continuous administration in a patient.

In certain embodiments, this disclosure provides an amylin to insulin molar dose ratio greater than 1:1, where amylin potency is comparable to currently used amylin agonists, for example pramlintide. In alternative embodiments, an amylin analog with greater potency than currently used agents is provided, wherein an amylin to insulin molar ratio of 1:1 corresponds to higher amylin activity than provided in current compositions. Definitions and characterization of “amylinomimetic”, or amylin analog responses necessary for such an analysis have been previously described (see e.g. U.S. Pat. No. 5,234,906; Young, A. (2005) Advances in Pharmacology 52: 151-171 2005).

In some embodiments, the amylin and insulin are provided in a molar ratio (amylin:insulin) of between about 1:1 to about 67:1, or between about 7:1 to about 67:1, or between about 1:1 and about 40:1, or between about 2.5:1 and about 35:1, or between about 5:1 and about 25:1, or between about 5:1 to about 10:1. In still other embodiments, an amylin composition is provided suitable for delivery to a patient at a dosage of at least about 5 micrograms per kilogram per day. In other embodiments, an amylin is provided suitable for delivery to a patient at a dosage of at least about 0.5, 1.0, 1.5, 2.0, 2.5, 3.0, 3.5, 4.0, or 4.5 micrograms per kilogram per day.

In some embodiments, an amylin analog polypeptide of the disclosure, without being co-formulated with insulin or an insulin derivative, is administered to a subject in combination with the insulin or an insulin derivative, i.e., as an adjunct to insulin therapy. In some embodiments, an amylin analog peptide of the disclosure, without being co-formulated with insulin or an insulin derivative, is administered to a subject in combination with meal-time insulin. In some embodiments, the subject has type 1 diabetes. In some embodiments, the subject has type 2 diabetes.

In some embodiments, an amylin analog polypeptide of the disclosure is co-administered to a human patient with insulin or an insulin derivative to provide a so-called dual-hormone “artificial pancreas” therapy. In some embodiments, an amylin analog polypeptide of the disclosure, without being co-formulated with the insulin or insulin derivative, is co-administered to a subject in combination with the insulin or insulin derivative to provide dual-hormone “artificial pancreas” therapy. In some embodiments, an amylin analog polypeptide of the disclosure is co-formulated with the insulin or insulin derivative and thus singly administered to a subject in combination with the insulin or insulin derivative to provide dual-hormone “artificial pancreas” therapy. In some embodiments, the artificial pancreas therapy includes rapid acting insulin or a rapid acting insulin derivative. In some embodiments, the artificial pancreas therapy includes a long acting or basal insulin or a long acting or basal insulin derivative.

Methods of Use

According to another embodiment, the disclosure relates to a method of treating metabolic disease or disorder in a subject in need of treatment, comprising providing the subject with an effective amount of an amylin analog polypeptide of the disclosure or a pharmaceutical composition thereof. Metabolic diseases or disorders include type 1 diabetes, type 2 diabetes, and obesity. Additionally, the disclosure relates to a method of effecting weight loss in a subject, including a diabetic subject, comprising providing the subject with an effective amount of an amylin analog polypeptide of the disclosure.

The disclosure also relates to an amylin analog polypeptide of the disclosure, or a pharmaceutical composition thereof, for use in the treatment of a metabolic disease or disorder in a subject in need of treatment, the use comprising providing the subject with an effective amount of the amylin analog peptide. Additionally, the disclosure relates to an amylin analog polypeptide of the disclosure, or a pharmaceutical composition thereof, for use in effecting weight loss in a subject, including a diabetic subject, comprising providing the subject with an effective amount of the amylin analog polypeptide.

Amylin analog polypeptides of the disclosure, like insulin, are provided (i.e., administered) to a diabetic subject to maintain, control, or reduce blood sugar concentrations in the subject. Diabetic subjects who are treated with an amylin analog polypeptide of the disclosure as an adjunct to insulin therapy are at risk of hypoglycemia (i.e., low blood sugar), particularly severe hypoglycemia. Accordingly, reducing the dose of meal time insulin for diabetic subjects upon treatment with an amylin analog polypeptide of the disclosure is intended to decrease the risk of hypoglycemia, particularly severe hypoglycemia.

Severe hypoglycemia, as used herein, refers to an episode of hypoglycemia requiring the assistance of another individual (including help administering oral carbohydrate) or requiring the administration of glucagon, intravenous glucose, or other medical intervention.

Accordingly, administration of an amylin analog polypeptide of the disclosure, as an adjunct to insulin therapy, particularly meal-time insulin therapy, generally requires a dose reduction in the meal-time insulin necessary to properly maintain healthy blood sugar concentrations in the subject. In other words, type 1 or type 2 diabetics who already self-administer meal-time insulin at a particular dose before commencing treatment with an amylin analog polypeptide of the disclosure, will reduce (for example, up to 25%, 50%, 75%, or 100%) the dose of meal-time insulin they continue to self-administer upon commencing treatment with an amylin analog polypeptide of the disclosure.

In some embodiments, the method comprises providing an amylin analog polypeptide of the disclosure or a pharmaceutical composition thereof, to a subject in need of treatment, via injection. In some embodiments, the method comprises providing an amylin analog polypeptide of the disclosure or a pharmaceutical composition thereof, formulated for oral administration, to a subject in need of treatment.

In some embodiments, the method comprises providing an amylin analog polypeptide of the disclosure or a pharmaceutical composition thereof, to a subject in need of treatment, via implantation. In some embodiments, the method comprises providing continuous delivery of an amylin analog polypeptide, to a subject in need of treatment, from an osmotic delivery device. The delivery device, such as an osmotic delivery device, comprises sufficient amylin analog polypeptide of the disclosure for continuous administration for up to 3 months, 6 months, 9 months, 12 months, 18 months or 24 months. As such, continuous administration of an amylin analog polypeptide of the disclosure via osmotic delivery device eliminates daily, or multiple daily dosing of existing amylin analog polypeptides, such as pramlintide. Diabetics who are treated with pramlintide must coordinate dosing of pramlintide before meals with meal-time insulin administered after meals. By contrast, diabetics who are treated with an amylin analog polypeptide of the disclosure via osmotic delivery device, receive continuous delivery of the amylin analog polypeptide and need only administer meal-time insulin at reduced doses.

The substantial steady-state delivery of the amylin analog polypeptide from the osmotic delivery device is continuous over an administration period. In some embodiments, the subject or patient is a human subject or human patient.

In some embodiments of the present disclosure, the administration period is, for example, at least about 3 months, at least about 3 months to about a year, at least about 4 months to about a year, at least about 5 months to about a year, at least about 6 months to about a year, at least about 8 months to about a year, at least about 9 months to about a year, at least about 10 months to about a year, at least about one year to about two years, at least about two years to about three years.

In further embodiments, the treatment methods of the present disclosure provide significant decrease in the subject's fasting plasma glucose concentration after implantation of the osmotic delivery device in the subject (relative to the subject's fasting plasma glucose concentration before implantation of the osmotic delivery device) that is achieved within about 7 days, 6 days, 5 days, 4 days, 3 days, 2 days, 1 day or less after implantation of the osmotic delivery device in the subject. The significant decrease in fasting plasma glucose is typically statistically significant as demonstrated by application of an appropriate statistical test or is considered significant for the subject by a medical practitioner. A significant decrease in fasting plasma glucose relative to the baseline before implantation is typically maintained over the administration period.

In some embodiments, the present disclosure relates to a method of treating a disease or condition in a subject in need of treatment. The method comprises providing continuous delivery of a drug from an osmotic delivery device, wherein substantial steady-state delivery of the drug at therapeutic concentrations is achieved in the subject. The substantial steady-state delivery of the drug from the osmotic delivery device is continuous over an administration period of at least about 3 months. The drug has a known or determined half-life in a typical subject. Humans are preferred subjects for the practice of the methods of the present disclosure. The present disclosure includes a drug effective for treatment of the disease or condition, as well as an osmotic delivery device comprising the drug for use in the present methods of treating the disease or condition in a subject in need of treatment. Advantages of the methods of the present disclosure include mitigation of peak-associated drug toxicities and attenuation of sub-optimal drug therapy associated with troughs.

In some embodiments, the substantial steady-state delivery of a drug at therapeutic concentrations is achieved within a period of about 1 month, 7 days, 5 days, 3 days or 1 day after implantation of the osmotic delivery device in the subject.

The disclosure also provides a method for promoting weight loss in a subject in need thereof, a method for treating excess weight or obesity in a subject in need thereof, and/or a method for suppressing appetite in a subject in need thereof. The method comprises providing delivery of an isolated amylin analog polypeptide. In some embodiments, the isolated amylin analog polypeptide is continuously delivered from an implantable osmotic delivery device. In some embodiments, substantial steady-state delivery of the amylin analog polypeptide from the osmotic delivery device is achieved and is substantially continuous over an administration period. In some embodiments, the subject is human.

The present disclosure includes an isolated amylin analog polypeptide, as well as an osmotic delivery device comprising an isolated amylin analog polypeptide for use in the present methods in a subject in need of treatment.

In embodiments of all aspects of the present disclosure relating to methods of treating a disease or condition in a subject, an exemplary osmotic delivery device comprises the following: an impermeable reservoir comprising interior and exterior surfaces and first and second open ends; a semi-permeable membrane in sealing relationship with the first open end of the reservoir, an osmotic engine within the reservoir and adjacent the semi-permeable membrane: a piston adjacent the osmotic engine, wherein the piston forms a movable seal with the interior surface of the reservoir, the piston divides the reservoir into a first chamber and a second chamber, the first chamber comprising the osmotic engine; a drug formulation or suspension formulation comprising the drug, wherein the second chamber comprises the drug formulation or suspension formulation and the drug formulation or suspension formulation is flowable; and a diffusion moderator inserted in the second open end of the reservoir, the diffusion moderator adjacent the suspension formulation. In preferred embodiments, the reservoir comprises titanium or a titanium alloy.

In embodiments of all aspects of the present disclosure relating to methods of treating a disease or condition in a subject, the drug formulation can comprise the drug and a vehicle formulation. Alternatively, suspension formulations are used in the methods and can, for example, comprise a particle formulation comprising the drug and a vehicle formulation. Vehicle formulations for use in forming the suspension formulations of the present disclosure can, for example, comprise a solvent and a polymer.

The reservoir of the osmotic delivery devices may, for example, comprise titanium or a titanium alloy.

In embodiments of all aspects of the present disclosure the implanted osmotic delivery device can be used to provide subcutaneous delivery.

In embodiments of all aspects of the present disclosure the continuous delivery can, for example, be zero-order, controlled continuous delivery.

In certain embodiments, a continuous administration of an amylin agonist is provided by an Animas® Vibe™ pump in association with a DexcomG4® PLATINUM continuous glucose monitoring system. In some embodiments, the pump administers both amylin and insulin simultaneously. In alternative embodiments, the pump delivers one or other of insulin or amylin, and another pump or device delivers the remaining agent.

Pharmaceutically Acceptable Compositions

According to another embodiment, the disclosure provides a composition comprising a compound, i.e., isolated polypeptide, of this disclosure or a pharmaceutically acceptable derivative thereof and a pharmaceutically acceptable carrier, adjuvant, or vehicle. The amount of compound in compositions of this disclosure is such that is effective to measurably activate one or more amylin and/or calcitonin receptors, in a biological sample or in a patient. In certain embodiments, the amount of compound in compositions of this disclosure is such that is effective to measurably activate human amylin 3 receptor (hAMY3) and/or human calcitonin receptor (hCTR), in the absence or presence of human serum albumin, in a biological sample or in a patient. In certain embodiments, a composition of this disclosure is formulated for administration to a patient in need of such composition. In some embodiments, a composition of this disclosure is formulated for injectable administration to a patient. In some embodiments, a composition of this disclosure is formulated for administration to a patient via an implantable delivery device such as an osmotic deliver device.

The terms “patient” or “subject” as used herein, refer to an animal, preferably a mammal, and most preferably a human.

A “pharmaceutically acceptable derivative” means any non-toxic salt, ester, salt of an ester or other derivative of a compound of this disclosure that, upon administration to a recipient, is capable of providing, either directly or indirectly, a compound of this disclosure or an inhibitorily active metabolite or residue thereof.

The isolated polypeptides of the disclosure (also referred to herein as “active compounds”), and derivatives, fragments, analogs and homologs thereof, can be incorporated into pharmaceutical compositions suitable for administration. Such compositions typically comprise the isolated polypeptide, or a pharmaceutically acceptable salt thereof, and a pharmaceutically acceptable carrier. As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the disclosure is formulated to be compatible with its intended route of administration. Examples of routes of administration include parenteral, e.g., intravenous, intradermal, subdermal, subcutaneous, oral (e.g., inhalation), transdermal (i.e., topical), transmucosal, rectal, or combinations thereof. In some embodiments, a pharmaceutical composition or an isolated polypeptide of the disclosure is formulated for administration by topical administration. In some embodiments, a pharmaceutical composition or an isolated polypeptide of the disclosure is formulated for administration by inhalation administration. In some embodiments, the pharmaceutical composition is formulated for administration by a device or other suitable delivery mechanism that is suitable for subdermal or subcutaneous implantation and delivers the pharmaceutical composition subcutaneously. In some embodiments, the pharmaceutical composition is formulated for administration by an implant device that is suitable for subdermal or subcutaneous implantation and delivers the pharmaceutical composition subcutaneously. In some embodiments, the pharmaceutical composition is formulated for administration by an osmotic delivery device, e.g., an implantable osmotic delivery device, that is suitable for subdermal or subcutaneous placement or other implantation and delivers the pharmaceutical composition subcutaneously. Solutions or suspensions used for parenteral application, intradermal application, subdermal application, subcutaneous application, or combinations thereof can include the following components: a sterile diluent such as water for injection, saline solution, fixed oils, polyethylene glycols, glycerine, propylene glycol or other synthetic solvents; antibacterial agents such as benzyl alcohol or methyl parabens, antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for case of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

Drug Particle Formulations

In some embodiments, provided herein is a pharmaceutical composition comprising any of the disclosed polypeptides formulated as a trifluoroacetate salt, acetate salt or hydrochloride salt. In some embodiments, provided is a pharmaceutical composition comprising any of the disclosed polypeptides formulated as a trifluoroacetate salt. In some embodiments, provided is a pharmaceutical composition comprising any of the disclosed polypeptides formulated as an acetate salt. In some embodiments, provided is a pharmaceutical composition comprising any of the disclosed polypeptides formulated as a hydrochloride salt.

Compounds, i.e., isolated polypeptides or pharmaceutically acceptable salts thereof, for use in the practice of the methods of the present disclosure are typically added to particle formulations, which are used to make polypeptide-containing particles that are uniformly suspended, dissolved or dispersed in a suspension vehicle to form a suspension formulation. In some embodiments, the amylin analog polypeptide is formulated in a particle formulation and converted (e.g., spray dried) to particles. In some embodiments, the particles comprising the amylin analog polypeptide are suspended in a vehicle formulation, resulting in a suspension formulation of vehicle and suspended particles comprising the amylin analog polypeptide.

Preferably, particle formulations are formable into particles using processes such as spray drying, lyophilization, desiccation, freeze-drying, milling, granulation, ultrasonic drop creation, crystallization, precipitation, or other techniques available in the art for forming particles from a mixture of components. In one embodiment of the disclosure the particles are spray dried. The particles are preferably substantially uniform in shape and size.

In some embodiments, the present disclosure provides drug particle formulations for pharmaceutical use. The particle formulation typically comprises a drug and includes one or more stabilizing component (also referred to herein as “excipients”). Examples of stabilizing components include, but are not limited to, carbohydrates, antioxidants, amino acids, buffers, inorganic compounds, and surfactants. The amounts of stabilizers in the particle formulation can be determined experimentally based on the activities of the stabilizers and the desired characteristics of the formulation, in view of the teachings of the present specification.

In any of the embodiments, the particle formulation may comprise about 50 wt % to about 90 wt % drug, about 50 wt % to about 85 wt % drug, about 55 wt % to about 90 wt % drug, about 60 wt % to about 90 wt % drug, about 65 wt % to about 85 wt % drug, about 65 wt % to about 90 wt % drug, about 70 wt % to about 90 wt % drug, about 70 wt % to about 85 wt % drug, about 70 wt % to about 80 wt % drug, or about 70 wt % to about 75 wt % drug.

Typically, the amount of carbohydrate in the particle formulation is determined by aggregation concerns. In general, the carbohydrate amount should not be too high so as to avoid promoting crystal growth in the presence of water due to excess carbohydrate unbound to drug.

Typically, the amount of antioxidant in the particle formulation is determined by oxidation concerns, while the amount of amino acid in the formulation is determined by oxidation concerns and/or formability of particles during spray drying.

Typically, the amount of buffer in the particle formulation is determined by pre-processing concerns, stability concerns, and formability of particles during spray drying. Buffer may be required to stabilize drug during processing, e.g., solution preparation and spray drying, when all stabilizers are solubilized.

Examples of carbohydrates that may be included in the particle formulation include, but are not limited to, monosaccharides (e.g., fructose, maltose, galactose, glucose, D-mannose, and sorbose), disaccharides (e.g., lactose, sucrose, trehalose, and cellobiose), polysaccharides (e.g., raffinose, melezitose, maltodextrins, dextrans, and starches), and alditols (acyclic polyols; e.g., mannitol, xylitol, maltitol, lactitol, xylitol sorbitol, pyranosyl sorbitol, and myoinsitol). Suitable carbohydrates include disaccharides and/or non-reducing sugars, such as sucrose, trehalose, and raffinose.

Examples of antioxidants that may be included in the particle formulation include, but are not limited to, methionine, ascorbic acid, sodium thiosulfate, catalase, platinum, ethylenediaminetetraacetic acid (EDTA), citric acid, cysteine, thioglycerol, thioglycolic acid, thiosorbitol, butylated hydroxanisol, butylated hydroxyltoluene, and propyl gallate. Further, amino acids that readily oxidize can be used as antioxidants, for example, cysteine, methionine, and tryptophan.

Examples of amino acids that may be included in the particle formulation include, but are not limited to, arginine, methionine, glycine, histidine, alanine, leucine, glutamic acid, iso-leucine, L-threonine, 2-phenylamine, valine, norvaline, proline, phenylalanine, tryptophan, serine, asparagines, cysteine, tyrosine, lysine, and norleucine. Suitable amino acids include those that readily oxidize, e.g., cysteine, methionine, and tryptophan.

Examples of buffers that may be included in the particle formulation include, but are not limited to, citrate, histidine, succinate, phosphate, maleate, tris, acetate, carbohydrate, and gly-gly. Suitable buffers include citrate, histidine, succinate, and tris.

Examples of inorganic compounds that may be included in the particle formulation include, but are not limited to, NaCl, Na2SO4, NaHCO3, KCl, KH2PO4, CaCl2, and MgCl2.

In addition, the particle formulation may include other stabilizers/excipients, such as surfactants and salts. Examples of surfactants include, but are not limited to, Polysorbate 20, Polysorbate 80, PLURONIC® (BASF Corporation. Mount Olive, N.J.) F68, and sodium dodecyl sulfate (SDS). Examples of salts include, but are not limited to, sodium chloride, calcium chloride, and magnesium chloride.

The particles are typically sized such that they can be delivered via an implantable osmotic delivery device. Uniform shape and size of the particles typically helps to provide a consistent and uniform rate of release from such a delivery device; however, a particle preparation having a non-normal particle size distribution profile may also be used. For example, in a typical implantable osmotic delivery device having a delivery orifice, the size of the particles is less than about 30%, more preferably is less than about 20%, more preferably is less than about than 10%, of the diameter of the delivery orifice. In an embodiment of the particle formulation for use with an osmotic delivery system, wherein the delivery orifice diameter of the implant is about 0.5 mm, particle sizes may be, for example, less than about 150 microns to about 50 microns. In an embodiment of the particle formulation for use with an osmotic delivery system, wherein the delivery orifice diameter of the implant is about 0.1 mm, particle sizes may be, for example, less than about 30 microns to about 10 microns. In one embodiment, the orifice is about 0.25 mm (250 microns) and the particle size is about 2 microns to about 5 microns.

Those of ordinary skill in the art will appreciate that a population of particles follow principles of particle size distribution. Widely used, art-recognized methods of describing particle size distributions include, for example, average diameters and D values, such as the DSO value, which is commonly used to represent the mean diameter of the range of the particle sizes of a given sample.

Particles of a particle formulation have diameters of between about 2 microns to about 150 micron, e.g., less than 150 microns in diameter, less than 100 microns in diameter, less than 50 microns in diameter, less than 30 microns in diameter, less than 10 microns in diameter, less than 5 microns in diameter, and about 2 microns in diameter. Preferably, particles have diameters of between about 2 microns and about 50 microns.

Particles of a particle formulation comprising an isolated amylin analog polypeptide have average diameters of between about 0.3 microns to about 150 microns. Particles of a particle formulation comprising an isolated amylin analog polypeptide have average diameters of between about 2 microns to about 150 microns, e.g., less than 150 microns in average diameter, less than 100 microns in average diameter, less than 50 microns in average diameter, less than 30 microns in average diameter, less than 10 microns in average diameter, less than 5 microns in average diameter, and about 2 microns in average diameter. In some embodiments, particles have average diameters of between about 0.3 microns and 50 microns, for example, between about 2 microns and about 50 microns. In some embodiments, the particles have an average diameter between 0.3 microns and 50 microns, for example, between about 2 microns and about 50 microns, where each particle is less than about 50 microns in diameter.

Typically, the particles of the particle formulations, when incorporated in a suspension vehicle, do not settle in less than about 3 months, preferably do not settle in less than about 6 months, more preferably do not settle in less than about 12 months, more preferably do not settle in less than about 24 months at delivery temperature, and most preferably do not settle in less than about 36 months at delivery temperature. The suspension vehicles typically have a viscosity of between about 5,000 to about 30,000 poise, preferably between about 8,000 to about 25,000 poise, more preferably between about 10,000 to about 20,000 poise. In one embodiment, the suspension vehicle has a viscosity of about 15,000 poise, plus or minus about 3,000 poise. Generally speaking, smaller particles tend to have a lower settling rate in viscous suspension vehicles than larger particles. Accordingly, micron- to nano-sized particles are typically desirable. In viscous suspension formulation, particles of about 2 microns to about 7 microns of the present disclosure will not settle for at least 20 years at room temperature based on simulation modeling studies. In an embodiment of the particle formulation of the present disclosure, for use in an implantable osmotic delivery device, comprises particles of sizes less than about 50 microns, more preferably less than about 10 microns, more preferably in a range from about 2 microns to about 7 microns.

In summary, disclosed polypeptides, or pharmaceutically acceptable salts thereof, are formulated into dried powders in solid state particles, which preserve maximum chemical and biological stability of the drug. Particles offers long-term storage stability at high temperature, and therefore, allows delivery to a subject of stable and biologically effective drug for extended periods of time. Particles are suspended in suspension vehicles for administration to patients.

Particle Suspensions in Vehicles

In one aspect, the suspension vehicle provides a stable environment in which the drug particle formulation is dispersed. The drug particle formulations are chemically and physically stable (as described above) in the suspension vehicle. The suspension vehicle typically comprises one or more polymer and one or more solvent that form a solution of sufficient viscosity to uniformly suspend the particles comprising the drug. The suspension vehicle may comprise further components, including, but not limited to, surfactants, antioxidants, and/or other compounds soluble in the vehicle.

The viscosity of the suspension vehicle is typically sufficient to prevent the drug particle formulation from settling during storage and use in a method of delivery, for example, in an implantable, osmotic delivery device. The suspension vehicle is biodegradable in that the suspension vehicle disintegrates or breaks down over a period of time in response to a biological environment, while the drug particle is dissolved in the biological environment and the active pharmaceutical ingredient (i.e., the drug) in the particle is absorbed.

In embodiments, the suspension vehicle is a “single-phase” suspension vehicle, which is a solid, semisolid, or liquid homogeneous system that is physically and chemically uniform throughout.

The solvent in which the polymer is dissolved may affect characteristics of the suspension formulation, such as the behavior of drug particle formulation during storage. A solvent may be selected in combination with a polymer so that the resulting suspension vehicle exhibits phase separation upon contact with the aqueous environment. In some embodiments of the disclosure, the solvent may be selected in combination with the polymer so that the resulting suspension vehicle exhibits phase separation upon contact with the aqueous environment having less than approximately about 10% water.

The solvent may be an acceptable solvent that is not miscible with water. The solvent may also be selected so that the polymer is soluble in the solvent at high concentrations, such as at a polymer concentration of greater than about 30%. Examples of solvents useful in the practice of the present disclosure include, but are not limited to, lauryl alcohol, benzyl benzoate, benzyl alcohol, lauryl lactate, decanol (also called decyl alcohol), ethyl hexyl lactate, and long chain (C8 to C24) aliphatic alcohols, esters, or mixtures thereof. The solvent used in the suspension vehicle may be “dry,” in that it has a low moisture content. Preferred solvents for use in formulation of the suspension vehicle include lauryl lactate, lauryl alcohol, benzyl benzoate, and mixtures thereof.

Examples of polymers for formulation of the suspension vehicles of the present disclosure include, but are not limited to, a polyester (e.g., polylactic acid and polylacticpolyglycolic acid), a polymer comprising pyrrolidones (e.g., polyvinylpyrrolidone having a molecular weight ranging from approximately 2,000 to approximately 1,000,000), ester or ether of an unsaturated alcohol (e.g., vinyl acetate), polyoxyethylenepolyoxypropylene block copolymer, or mixtures thereof. Polyvinylpyrrolidone can be characterized by its K-value (e.g., K-17), which is a viscosity index. In one embodiment, the polymer is polyvinylpyrrolidone having a molecular weight of 2,000 to 1,000,000. In a preferred embodiment, the polymer is polyvinylpyrrolidone K-17 (typically having an approximate average molecular weight range of 7,900-10,800). The polymer used in the suspension vehicle may include one or more different polymers or may include different grades of a single polymer. The polymer used in the suspension vehicle may also be dry or have a low moisture content.

Generally speaking, a suspension vehicle for use in the present disclosure may vary in composition based on the desired performance characteristics. In one embodiment, the suspension vehicle may comprise about 40 wt % to about 80 wt % polymer(s) and about 20 wt % to about 60 wt % solvent(s). Preferred embodiments of a suspension vehicle include vehicles formed of polymer(s) and solvent(s) combined at the following ratios: about 25 wt % solvent and about 75 wt % polymer; about 50 wt % solvent and about 50 wt % polymer; about 75 wt % solvent and about 25 wt % polymer. Accordingly, in some embodiments, the suspension vehicle may comprise selected components and in other embodiments consist essentially of selected components.

The suspension vehicle may exhibit Newtonian behavior. The suspension vehicle is typically formulated to provide a viscosity that maintains a uniform dispersion of the particle formulation for a predetermined period of time. This helps facilitate making a suspension formulation tailored to provide controlled delivery of the drug contained in the drug particle formulation. The viscosity of the suspension vehicle may vary depending on the desired application, the size and type of the particle formulation, and the loading of the particle formulation in the suspension vehicle. The viscosity of the suspension vehicle may be varied by altering the type or relative amount of the solvent or polymer used.

The suspension vehicle may have a viscosity ranging from about 100 poise to about 1,000,000 poise, preferably from about 1,000 poise to about 100,000 poise. In preferred embodiments, the suspension vehicles typically have a viscosity, at 33° C., of between about 5,000 to about 30,000 poise, preferably between about 8,000 to about 25,000 poise, more preferably between about 10,000 to about 20,000 poise. In one embodiment, the suspension vehicle has a viscosity of about 15,000 poise, plus or minus about 3.000 poise, at 33° C. The viscosity may be measured at 33′ C., at a shear rate of 10-4/sec, using a parallel plate rheometer.

The suspension vehicle may exhibit phase separation when contacted with the aqueous environment; however, typically the suspension vehicle exhibits substantially no phase separation as a function of temperature. For example, at a temperature ranging from approximately 0° C. to approximately 70° C. and upon temperature cycling, such as cycling from 4° C. to 37° C. to 4° C., the suspension vehicle typically exhibits no phase separation.

The suspension vehicle may be prepared by combining the polymer and the solvent under dry conditions, such as in a dry box. The polymer and solvent may be combined at an elevated temperature, such as from approximately 40° C. to approximately 70° C., and allowed to liquefy and form the single phase. The ingredients may be blended under vacuum to remove air bubbles produced from the dry ingredients. The ingredients may be combined using a conventional mixer, such as a dual helix blade or similar mixer, set at a speed of approximately 40 rpm. However, higher speeds may also be used to mix the ingredients. Once a liquid solution of the ingredients is achieved, the suspension vehicle may be cooled to room temperature. Differential scanning calorimetry (DSC) may be used to verify that the suspension vehicle is a single phase. Further, the components of the vehicle (e.g., the solvent and/or the polymer) may be treated to substantially reduce or substantially remove peroxides (e.g., by treatment with methionine; see, e.g., U.S., Patent Application Publication No. 2007-0027105).

The drug particle formulation is added to the suspension vehicle to form a suspension formulation. In some embodiments, the suspension formulation may comprise a drug particle formulation and a suspension vehicle and in other embodiments consist essentially of a drug particle formulation and a suspension vehicle.

The suspension formulation may be prepared by dispersing the particle formulation in the suspension vehicle. The suspension vehicle may be heated and the particle formulation added to the suspension vehicle under dry conditions. The ingredients may be mixed under vacuum at an elevated temperature, such as from about 40° C. to about 70° C. The ingredients may be mixed at a sufficient speed, such as from about 40 rpm to about 120 rpm, and for a sufficient amount of time, such as about 15 minutes, to achieve a uniform dispersion of the particle formulation in the suspension vehicle. The mixer may be a dual helix blade or other suitable mixer. The resulting mixture may be removed from the mixer, sealed in a dry container to prevent water from contaminating the suspension formulation, and allowed to cool to room temperature before further use, for example, loading into an implantable, drug delivery device, unit dose container, or multiple-dose container.

The suspension formulation typically has an overall moisture content of less than about 10 wt %, preferably less than about 5 wt %, and more preferably less than about 4 wt %.

In preferred embodiments, the suspension formulations of the present disclosure are substantially homogeneous and flowable to provide delivery of the drug particle formulation from the osmotic delivery device to the subject.

In summary, the components of the suspension vehicle provide biocompatibility. Components of the suspension vehicle offer suitable chemico-physical properties to form stable suspensions of drug particle formulations. These properties include, but are not limited to, the following: viscosity of the suspension; purity of the vehicle; residual moisture of the vehicle; density of the vehicle; compatibility with the dry powders: compatibility with implantable devices: molecular weight of the polymer, stability of the vehicle; and hydrophobicity and hydrophilicity of the vehicle. These properties can be manipulated and controlled, for example, by variation of the vehicle composition and manipulation of the ratio of components used in the suspension vehicle.

The suspension formulations described herein may be used in an implantable, osmotic delivery device to provide zero-order, continuous, controlled, and sustained delivery of a compound over an extended period of time, such as over weeks, months, or up to about one year or more. Such an implantable osmotic delivery device is typically capable of delivering the suspension formulation, comprising the drug, at a desired flow rate over a desired period of time. The suspension formulation may be loaded into the implantable, osmotic delivery device by conventional techniques.

Implantable Delivery

A dose and delivery rate can be selected to achieve a desired blood concentration of a drug generally within less than about 6 half-lives of the drug within the subject after implantation of the device. The blood concentration of the drug is selected to give the optimal therapeutic effects of the drug while avoiding undesirable side effects that may be induced by excess concentration of the drug, while at the same time avoiding peaks and troughs that may induce side effects associated with peak or trough plasma concentrations of the drug.

The implantable, osmotic delivery device typically includes a reservoir having at least one orifice through which the suspension formulation is delivered. The suspension formulation may be stored within the reservoir. In a preferred embodiment, the implantable, drug delivery device is an osmotic delivery device, wherein delivery of the drug is osmotically driven. Some osmotic delivery devices and their component parts have been described, for example, the DUROS® delivery device or similar devices (see, e.g., U.S. Pat. Nos. 5,609,885; 5,728,396; 5,985,305; 5,997,527; 6,113,938; 6,132,420; 6,156,331; 6,217,906; 6,261,584; 6,270,787; 6,287,295; 6,375,978; 6,395,292; 6,508,808; 6,544,252; 6,635,268; 6,682,522; 6,923,800; 6,939,556; 6,976,981; 6,997,922; 7,014,636; 7,207,982; and 7,112,335; 7,163,688; U.S. Patent Publication Nos. 2005/0175701, 2007/0281024, 2008/0091176, and 2009/0202608).

The osmotic delivery device typically consists of a cylindrical reservoir which contains the osmotic engine, piston, and drug formulation. The reservoir is capped at one end by a controlled-rate, semi-permeable membrane and capped at the other end by a diffusion moderator through which suspension formulation, comprising the drug, is released from the drug reservoir. The piston separates the drug formulation from the osmotic engine and utilizes a seal to prevent the water in the osmotic engine compartment from entering the drug reservoir. The diffusion moderator is designed, in conjunction with the drug formulation, to prevent body fluid from entering the drug reservoir through the orifice.

The osmotic device releases a drug at a predetermined rate based on the principle of osmosis. Extracellular fluid enters the osmotic delivery device through a semi-permeable membrane directly into a salt engine that expands to drive the piston at a slow and even delivery rate. Movement of the piston forces the drug formulation to be released through the orifice or exit port at a predetermined shear rate. In one embodiment of the present disclosure, the reservoir of the osmotic device is loaded with a suspension formulation wherein the device is capable of delivering the suspension formulation to a subject over an extended period of time (e.g., about 1, about 3, about 6, about 9, about 10, and about 12 months) at a pre-determined, therapeutically effective delivery rate.

The release rate of the drug from the osmotic delivery device typically provides a subject with a predetermined target dose of a drug, for example, a therapeutically effective daily dose delivered over the course of a day; that is, the release rate of the drug from the device, provides substantial steady-state delivery of the drug at a therapeutic concentration to the subject.

Typically, for an osmotic delivery device, the volume of a beneficial agent chamber comprising the beneficial agent formulation is between about 100 μl to about 1000 μl, more preferably between about 120 μl and about 500 μl, more preferably between about 150 μl and about 200 μl.

Typically, the osmotic delivery device is implanted within the subject, for example, subdermally or subcutaneously to provide subcutaneous drug delivery. The device(s) can be implanted subdermally or subcutaneously into either or both arms (e.g., in the inside, outside, or back of the upper arm) or the abdomen. Preferred locations in the abdominal area are under the abdominal skin in the area extending below the ribs and above the belt line. To provide a number of locations for implantation of one or more osmotic delivery device within the abdomen, the abdominal wall can be divided into 4 quadrants as follows: the upper right quadrant extending at least 2-3 centimeters below the right ribs, e.g., at least about 5-8 centimeters below the right ribs, and at least 2-3 centimeters to the right of the midline, e.g., at least about 5-8 centimeters to the right of the midline; the lower right quadrant extending at least 2-3 centimeters above the belt line, e.g., at least about 5-8 centimeters above the belt line, and at least 2-3 centimeters to the right of the midline, e.g., at least about 5-8 centimeters to the right of the midline; the upper left quadrant extending at least 2-3 centimeters below the left ribs, e.g., at least about 5-8 centimeters below the let ribs, and at least 2-3 centimeters to the left of the midline, e.g., at least about 5-8 centimeters to the left of the midline: and the lower left quadrant extending at least 2-3 centimeters above the belt line, e.g., at least about 5-8 centimeters above the belt line, and at least 2-3 centimeters to the left of the midline, e.g., at least about 5-8 centimeters to the left of the midline. This provides multiple available locations for implantation of one or more devices on one or more occasions. Implantation and removal of osmotic delivery devices are generally carried out by medical professionals using local anesthesia (e.g., lidocaine).

Termination of treatment by removal of an osmotic delivery device from a subject is straightforward, and provides the important advantage of immediate cessation of delivery of the drug to the subject.

Preferably, the osmotic delivery device has a fail-safe mechanism to prevent an inadvertent excess or bolus delivery of drug in a theoretical situation like the plugging or clogging of the outlet (diffusion moderator) through which the drug formulation is delivered. To prevent an inadvertent excess or bolus delivery of drug the osmotic delivery device is designed and constructed such that the pressure needed to partially or wholly dislodge or expel the diffusion moderator from the reservoir exceeds the pressure needed to partially or wholly dislodge or expel the semi-permeable membrane to the extent necessary to de-pressurize the reservoir. In such a scenario, pressure would build within the device until it would push the semi-permeable membrane at the other end outward, thereby releasing the osmotic pressure.

The osmotic delivery device would then become static and no longer deliver the drug formulation provided that the piston is in a sealing relationship with the reservoir.

The suspension formulations may also be used in infusion pumps, for example, the ALZET® (DURECT Corporation, Cupertino, Calif.) osmotic pumps which are miniature, infusion pumps for the continuous dosing of laboratory animals (e.g., mice and rats).

Kits

The present disclosure also provides a kit for treating type 1 diabetes, comprising an amylin analog, in a unit dosage that is (i) at least 5 μg per kilogram of the subject per day or (ii) at or greater than the ED75 dose of the amylin analog. In some embodiments, the kits provide the amylin analog in a form that is compatible with continuous administration.

In certain embodiments, the kit comprises a pharmaceutical composition of the disclosure. In some embodiments, the kit comprises a pharmaceutical composition comprising an amylin analog of the disclosure and a pharmaceutically acceptable carrier, adjuvant, or vehicle.

In certain embodiments, the kit comprises an implantable, osmotic delivery device of the disclosure. In certain embodiments, the kit comprises an amylin analog of the disclosure or a pharmaceutical composition thereof.

In some embodiments, the kit further comprises an insulin.

In certain embodiments, the kit includes a sealed container approved for the storage of pharmaceutical compositions, the container containing one of the above-described pharmaceutical compositions. In some embodiments, the sealed container minimizes the contact of air with the ingredients. An instruction for the use of the composition and the information about the composition are to be included in the kit.

The kits provided herein may include prescribing information, for example, to a patient or health care provider, or as a label in a packaged pharmaceutical formulation. Prescribing information may include, for example, efficacy, dosage and administration, contraindication and adverse reaction information pertaining to the pharmaceutical formulation.

A kit provided herein can be designed for conditions necessary to properly maintain the components housed therein (e.g., refrigeration or freezing). A kit can contain a label or packaging insert including identifying information for the components therein and instructions for their use (e.g., dosing parameters, clinical pharmacology of the active ingredient(s), including mechanism(s) of action, pharmacokinetics and pharmacodynamics, adverse effects, contraindications, etc.).

Each component of the kit can be enclosed within an individual container, and all of the various containers can be within a single package. Labels or inserts can include manufacturer information such as lot numbers and expiration dates. The label or packaging insert can be, e.g., integrated into the physical structure housing the components, contained separately within the physical structure, or affixed to a component of the kit.

### EXAMPLE

The following example is put forth so as to provide those of ordinary skill in the art with a complete disclosure and description of how to practice the methods of the present disclosure and is not intended to limit the scope of what the inventors regard as the invention. Efforts have been made to ensure accuracy with respect to numbers used (e.g., amounts, concentrations, and percent changes) but some experimental errors and deviations should be accounted for. Unless indicated otherwise, temperature is in degrees Centigrade and pressure is at or near atmospheric.

### Example 1: Preclinical Assessment of Constant High Amylin Analog Activity in Association with Insulin Therapy for Treatment of Type 1 Diabetes

### Methods

### Animal Model

Male Wistar rats are implanted intraperitoneally (sensor in abdominal aorta) with an HD-XG continuous glucose monitor (Data Sciences, St Paul, Minn.) (Brockway, Tiesma et al. 2015). This system allows up to 8 weeks of continuous capture of blood glucose concentrations.

Upon recovery from surgery and resumption of normal food intake, capture of glucose readings begins so as to include at least 4 days of non-diabetic record for each rat. To induce insulinopenic (type 1) diabetes, rats are fasted overnight and administered an intravenous dose of streptozotocin (STZ) of 60 mg/kg (Gajdosik, Gajdosikova et al. 1999). Glucose readings from the telemetry system are used to enhance survival post-STZ, by s.c. glucose supplementation in the event of hypoglycemia that often accompanies an initial release of insulin following β-cell toxicity. The telemetry is then used to affirm hyperglycemia (mean plasma glucose >300 mg/dL), and to determine if a second STZ treatment is necessary for rats that are not sufficiently hyperglycemic.

Upon becoming hyperglycemic, animals are treated daily with a sliding-scale of a long-acting insulin (insulin detemir, Levemir NOVO) so as to eliminate urinary ketones, but maintain 5% glycosuria, as determined by daily KETOSTIX and GLUCOSTIX testing, respectively (Young, Crocker et al. 1991).

### Treatments

Animals are assigned to one of 3 cohorts of insulin dosing (n=5/cohort). One cohort is defined as that necessary to attain −ve urinary ketones and +ve urinary glucose, as just described, but not greater than a total dose of 2 U/day. A second cohort is a high insulin dose cohort, 3× greater than cohort 1. A third cohort is one where insulin dose is 50% of that of cohort 1.

Following at least 1 week of insulin dose stabilization, animals are entered into each of 5 supplemental treatments comprising administration of the long-acting amylin agonist, compound A2, at doses of 1, 3, 10, 30 or 100 μg/day as a single daily injection, in addition to the fixed daily Levemir dose. (Alternatively, these studies could utilize relatively short-acting pramlintide as an amylin analog.) The 0% of compound A2 is 32-37 hours in the rat, similar to that of albumin-bound insulin detemir. Daily dosing of each therefore maintains relatively constant concentrations of each, and a relatively ratio of concentrations.

Each dose level of compound A2 is maintained for 1 week, during which glucose data is captured via telemetry. The order in which doses of compound A2 are changed is determined by a 5×5 orthogonal latin square. That is, each animal receives each dose of compound A2, but in an order that is unique relative to that of the other 4 animals in the same insulin cohort. This treatment balances out time-dependent or order-dependent changes in metabolic status of each animal, such as regeneration of insulin secretory capacity, and accommodation to the effects of an amylin agonist. An example of such a latin square is shown in Table 3.

TABLE 3
Order of each of 5 treatments (1-5) shown top-to-
bottom in columns for each of 5 different rats.

| | | | | |
|---|---|---|---|---|
| 1 | 2 | 3 | 4 | 5 |
| 2 | 3 | 5 | 1 | 4 |
| 3 | 5 | 4 | 2 | 1 |
| 4 | 1 | 2 | 5 | 3 |
| 5 | 4 | 1 | 3 | 2 |

### Data Analysis

Plasma glucose values from the final 4 days of each insulin/A2 combination are aggregated and analyzed according to frequency of occurrence (cumulative distributions). Analogous to the time-in-range (TIR) assessment of clinical benefit (sometimes termed “clinical utility”) in human diabetes trials (Beck, Bergenstal et al. 2018), glucose values are categorized within bins (<70, 70-180, and >180 mg/dL). Further cuts (>250 mg/dL) are also made. Parametric descriptors of glucose values are also derived for each combination (mean and SD values, linear and logarithmic, where data are not normal but are log-normal).

### Data Interpretation

Higher benefit (i.e., utility) is indicated when TIR is greatest, with the proviso that values below 70 mg/dL are not more frequent. Higher benefit is also indicated when the SD for the distribution of glucose values is least.

### Potential Results: Distribution of Pre- and Post-STZ Blood Glucose Values

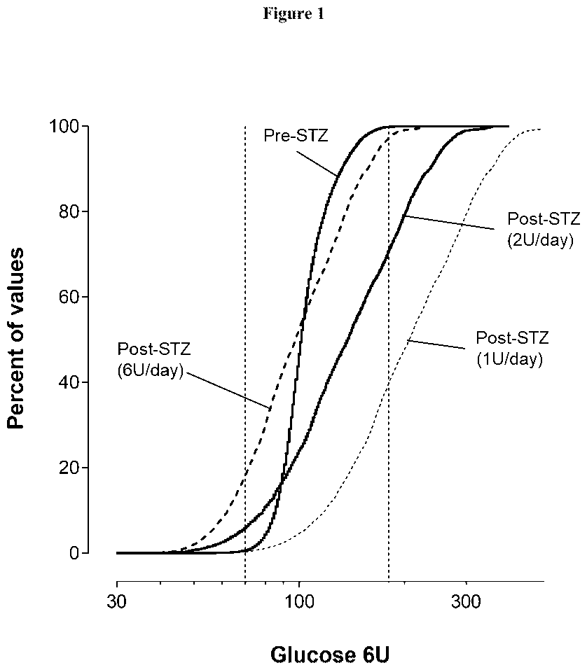

The cumulative distribution of blood glucose values before and after STZ treatment is shown in FIG. 1. The range 70-180 mg/dL is shown by the vertical dotted lines, 98.5% of pre-STZ values fell within the range (TIR=98.8%). The value post-STZ treated with 2 U/day Levemir was 64.9%; 5.8% of values were <70 mg/dL.

### Hazard Indices

Blood Glucose Concentrations: Time Below 70 mg/dL (hypoglycemic)

Higher insulin doses, in the absence of compound A2, markedly increase the proportion of time spent below 70 mg/dL. Higher numerical values indicate greater intensity of the hazard indicia.

TABLE 4
Ratios of insulin (U/day) and compound A2 (μg/kg/day)
separately co-administered. Values in each cell correspond
to “hazard indices” wherein higher values are
ascribed to less efficacious ratios that promote hypoglycemia.

| units (U) | A2 administration in μg/kg/day | | | | | |
|---|---|---|---|---|---|---|
| insulin per day | 0 | 1 | 3 | 10 | 30 | 100 |
| Pre STZ | 0.6 | | | | | |
| 1 U/day | 1.2 | 1.2 | 1.2 | 1.2 | 1.1 | 1.0 |
| 2 U/day | 5.8 | 5.8 | 5.8 | 5.7 | 5.5 | 4.6 |
| 6 U/day | 18.3 | 18.3 | 18.2 | 17.9 | 17.2 | 14.6 |

Note from Table 4 that relatively high doses of long acting insulin (6 U/day) correspond to less efficacious ratios that permit relatively high “hazard indices” for the onset of hypoglycemia.

Blood Glucose Concentrations: Time Above 180 mg/dL (Hyperglycemic)

Lower insulin doses, in general, are associated with a greater proportion of glucose values being greater than 180 mg/dL. This proportion is reduced with concomitant administration of compound A2. Higher numerical values indicate greater intensity of the hazard indicia.

TABLE 5
Ratios of insulin (U/day) and compound A2
(μg/kg/day) separately co-administered

| units (U) | A2 administration in μg/kg/day | | | | | |
|---|---|---|---|---|---|---|
| insulin per day | 0 | 1 | 3 | 10 | 30 | 100 |
| Pre STZ | 0.2 | | | | | |
| 1 U/day | 60.2 | 59.8 | 59.9 | 56.0 | 47.6 | 18.1 |
| 2 U/day | 29.4 | 29.2 | 28.8 | 27.3 | 23.2 | 8.8 |
| 6 U/day | 2.8 | 2.8 | 2.7 | 2.6 | 2.2 | 0.8 |

Note from Table 5 that relatively low doses of long acting insulin (6 U/day) and low doses of compound A2 (0-10 μg/kg/day) correspond to less efficacious ratios that permit relatively high “hazard indices” for the onset of hyperglycemia.

### Benefit Indices

### Time in Range

Proportion of time spent above 70, but below 180 mg/dL is shown below. The highest number occurred with high insulin dosing and high compound A2 dosing. However, the high insulin dosing also carried a much higher risk of hypoglycemia. Therefore, a time-in-range index of benefit is generated (Table 6). Higher numerical values indicate greater intensity of the benefit indicia (i.e., where TIR is greatest).

TABLE 6
Ratios of insulin (U/day) and compound A2
(μg/kg/day) separately co-administered

| units (U) | A2 administration in μg/kg/day | | | | | |
|---|---|---|---|---|---|---|
| insulin per day | 0 | 1 | 3 | 10 | 30 | 100 |
| Pre STZ | 99.2 | | | | | |
| 1 U/day | 39.5 | 39.0 | 39.9 | 42.8 | 51.3 | 81.0 |
| 2 U/day | 64.9 | 65.0 | 65.5 | 67.0 | 71.3 | 86.5 |
| 6 U/day | 78.9 | 79.0 | 79.1 | 79.5 | 80.6 | 84.5 |

Note from Table 6 that relatively high doses of compound A2 (100 μg/kg/day), even when separately co-administered with low doses of long acting insulin (1 U/day) correspond to more efficacious ratios that permit relatively high “benefit indices” for time-in-range (TIR, i.e., the time during which a type 1 diabetic patient maintains blood glucose concentrations of approximately 70 mg/dL to 180 mg/dL).

### Hypo Avoidance Index

The time-in-range benefit index of Table 6, often used to describe the benefit of different therapeutic interventions, fails to accommodate the reality that the hazards of hyperglycemia and hypoglycemia are not necessarily symmetrical. Prolonged periods of hyperglycemia promote irreversible microvascular disease, and are to be avoided in general. Hyperglycemia also invokes osmotic and electrolyte disturbances and is to be avoided on that basis. However, excursions into the hyperglycemic range for periods of an hour, for example, do not convey the hazard as excursions into the hypoglycemic range of the same duration. The hazard of hypoglycemia is rarely cytotoxic, but is more typically contextual, and relates to loss of volitional control in situations where control is necessary. Examples include driving, operating machinery, or during childcare. To accommodate a supreme need to avoid acute hypoglycemia, an index is constructed here to reflect the asymmetry of hazards. This benefit index is weighted such that the hazard of being below 70 mg/dL is 5× greater than the hazard of being over 180 mg/dL. Higher numerical values indicate greater intensity of the benefit indicia (i.e., where TIR is greatest, accounting for the weighted reduction of hazards). The most beneficial ratios now shift to lower insulin dosing and high fixed dosing of compound A2, in contrast to the pattern shown in Table 6 for time-in-range:

TABLE 7
Ratios of insulin (U/day) and compound A2
(μg/kg/day) separately co-administered

| units (U) | A2 administration in μg/kg/day | | | | | |
|---|---|---|---|---|---|---|
| insulin per day | 0 | 1 | 3 | 10 | 30 | 100 |
| Pre STZ | 98.8 | | | | | |
| 1 U/day | 33.8 | 34.2 | 35.1 | 38.1 | 46.8 | 77.1 |
| 2 U/day | 41.6 | 41.9 | 42.4 | 44.2 | 49.5 | 68.0 |
| 6 U/day | 5.7 | 5.9 | 6.3 | 7.7 | 11.8 | 26.0 |

Note from Table 7 that relatively high doses of compound A2 (100 μg/kg/day) and low doses of long acting insulin (1 U/day) correspond to more efficacious ratios that permit relatively high “benefit indices” for TIR.

### Summary

Combinations of a fixed dose of an amylin analog and variable dosing of insulin affect the distributions of glucose values differently. Individuals with insulin-dependent diabetes (type 1 diabetes, and end-stage type 2 diabetes) need to balance minimizing the long-term hazard of microvascular disease from sustained hyperglycemia versus the acute hazards of hypoglycemia, which include not only its corporal effects, but also the situational hazards invoked during neuroglycopenia.

If a higher weighting is applied to the hazard of hypoglycemia, it is apparent that the greatest benefit is observed with reduced doses of insulin in combination with high (supraphysiologic) amylin activity.

### REFERENCES

Beck, R. W., R. M. Bergenstal, T. D. Riddlesworth, C. Kollman, Z. Li, A. S. Brown and K. L. Close (2018). “Validation of Time in Range as an Outcome Measure for Diabetes Clinical Trials.” Diabetes Care.
Brockway, R., S. Tiesma, H. Bogie, K. White, M. Fine, L. O'Farrell, M. Michael, A. Cox and T. Coskun (2015). “Fully Implantable Arterial Blood Glucose Device for Metabolic Research Applications in Rats for Two Months.” Journal of Diabetes Science and Technology 9(4): 771-781.
Gajdosik, A., A. Gajdosikova, M. Stefek, J. Navarova and R. Hozova (1999). “Streptozotocin-induced experimental diabetes in male Wistar rats.” Gen Physiol Biophys 18 Spec No: 54-62.
Young, A. A., L. B. Crocker, D. Wolfe-Lopez and G. J. Cooper (1991). “Daily amylin replacement reverses hepatic glycogen depletion in insulin-treated streptozotocin diabetic rats.” FEBS Lett 287(1-2): 203-205.
Young, A. A., W. Vine, B. R. Gedulin, R. Pittner, S. Janes, L. S. L. Gaeta, A. Percy, C. X. Moore, J. E. Koda, T. J. Rink and K. Beaumont (1996). “Preclinical pharmacology of pramlintide in the rat: comparisons with human and rat amylin.” Drug Dev Res 37(4): 231-248.

### Other Embodiments

While the methods of the disclosure have been described in conjunction with the detailed description thereof, the foregoing description is intended to illustrate and not limit the scope of these methods, which are defined by the scope of the appended claims. Other aspects, advantages, and modifications are within the scope of the following claims.

1. A method of treating type 1 diabetes in a human subject, comprising administering to the subject a pharmaceutical composition comprising an amylin analog at a therapeutically effective dose:
(i) of at least 5 μg per kilogram of the subject per day; or
(ii) that is at or greater than the ED75 dose of the amylin analog.

2. The method of claim 1, further comprising continuously maintaining a concentration of the amylin analog in the subject that is at or greater than the EC75 dose of the amylin analog.

3. The method of claim 1, comprising continuous administration of the amylin analog.

4. The method of claim 1, comprising continuous administration of the amylin analog via an implantable drug delivery device.

5. The method of claim 4, wherein the implantable drug delivery device is an osmotic drug delivery device.

6. The method of claim 1, comprising continuous administration of the amylin analog via a non-implantable drug delivery device.

7. The method of claim 1, comprising continuous administration of the amylin analog via once weekly injection.

8. The method of claim 1, wherein the amylin analog is pramlintide.

9. The method of claim 1, wherein the amylin analog is compound A2 (SEQ ID NO:2).

10. The method of claim 1, further comprising separate administration of an insulin.

11. The method of claim 2, comprising continuous administration of the amylin analog.

12. The method of claim 2, comprising continuous administration of the amylin analog via an implantable drug delivery device.

13. The method of claim 12, wherein the implantable drug delivery device is an osmotic drug delivery device.

14. The method of claim 2, comprising continuous administration of the amylin analog via a non-implantable drug delivery device.

15. The method of claim 2, comprising continuous administration of the amylin analog via once weekly injection.

16. The method of claim 2, wherein the amylin analog is pramlintide.

17. The method of claim 2, wherein the amylin analog is compound A2 (SEQ ID NO:2).

18. The method of claim 2, further comprising separate administration of an insulin.


SEQUENCE LISTING (5 sequences)

SEQ ID NO: 1 - protein, 37 aa, Artificial Sequence
  source: /note=“Description of Artificial Sequence Synthetic polypeptide”
  DISULFIDE (2)..(7): DISULFIDE
  MOD_RES (16)..(16): D-Lys(gammaGlu-gammaGlu-CO(CH2)14CH3)
SCNTSTCATQRLANEXHKSSNNFGPILPPTKVGSETY

SEQ ID NO: 2 - protein, 37 aa, Artificial Sequence
  source: /note=“Description of Artificial Sequence Synthetic polypeptide”
  MOD_RES (1)..(1): Lys(gammaGlu-gammaGlu-CO(CH2)18CO2H)
  DISULFIDE (2)..(7): DISULFIDE
KCNTSTCATQRLANELHKSSNNFGPILPPTKVGSETY

SEQ ID NO: 3 - protein, 37 aa, Artificial Sequence; identical to SEQ ID NO: 2
  source: /note=“Description of Artificial Sequence Synthetic polypeptide”
  MOD_RES (1)..(1): Lys(gammaGlu-gammaGlu-CO(CH2)16CO2H)
  DISULFIDE (2)..(7): DISULFIDE

SEQ ID NO: 4 - protein, 37 aa, Artificial Sequence
  source: /note=“Description of Artificial Sequence Synthetic polypeptide”
  MOD_RES (1)..(1): Lys(gammaGlu-CO(CH2)16CO2H)
  DISULFIDE (2)..(7): DISULFIDE
KCNTSTCATSRLANFLQKSSNNFGPILPPTKVGSETY

SEQ ID NO: 5 - protein, 37 aa, Homo sapiens [sequence omitted]